SEQUENCE LISTING (12 sequences)

SEQ ID NO: 1 - DNA, 706 bp, Homo Sapiens
gagccgaactccaagatgggaggcaagctcagcaagaagaagaagggctacaatgtgaacgacgagaaagccaaggagaaagacaagaaggccgagggcgcggcgacggaagaggaggggaccccgaaggagagtgagccccaggcggccgcagagcccgccgaggccaaggagggcaaggagaagcccgaccaggacgccgagggcaaggccgaggagaaggagggcgagaaggacgcggcggctgccaaggaggaggccccgaaggcggagcccgagaagacggagggcgcggcagaggccaaggctgagcccccgaaggcgcccgagcaggagcaggcggcccccggccccgctgcgggcggcgaggcccccaaagctgctgaggccgccgcggccccggccgagagcgcggcccctgccgccggggaggagcccagcaaggaggaaggggaacccaaaaagactgaggcgcccgcagctcctgccgcccaggagaccaaaagtgacggggccccagcttcagactcaaaacccggcagctcggaggctgccccctcttccaaggagacccccgcagccacggaagcgcctagttccacacccaaggcccagggccccgcagcctctgcagaagagcccaagccggtggaggccccggcagctaattccgaccaaaccgtaaccgtgaaagagtgacaaggac

SEQ ID NO: 2 - DNA, 3369 bp, Homo Sapiens
gctgaggaaggaatggcagacatggaggtgggtgagtggcagggacagatcaaggttcagttggaaccaaggcctggttaggtcaggaatcagatgtactcctttctgccttcaggtgctctgcttctagtccgccagaaatgggatgcatctggatgccaaggcaatcctgggcccatgccacctccttgagatccaggtgtcttatatcacagcatccctttcctattctcagaacagggtccaagctgtggacaagctttcccaggtctcctttccttttgtaggcagagatcaagaatggtgcttgtggcggcagagggcatggaatgaggaaccaggcatctccaactccttctgcctttccctacaggaatgggatgctgggcccgggaggtgctggtccccgaggggcccttgtaccgcgtggctggcacagctgtctccatctcctgcaatgtgaccggctatgagggccctgcccagcagaacttcgagtggttcctgtataggcccgaggccccagatactgcactgggcattgtcagtaccaaggatacccagttctcctatgctgtcttcaagtcccgagtggtggcgggtgaggtgcaggtgcagcgcctacaaggtgatgccgtggtgctcaagattgcccgcctgcaggcccaggatgccggcatttatgagtgccacaccccctccactgatacccgctacctgggcagctacagcggcaaggtggagctgagaggtactgggccctgggacggggtggagccacacaccctctcaaggcagaaagtcaaggccacaagtggcctatctagttgccccttaggttttgtagccaaggaacttgaggcccaaagaggttaagtgatttgcacaaagtcacccaataactcagtggctgatgtgggactcaaacccagagttcctcacccaggccagtgctcatcttatcatattaagacccctcctgggagaagaagtgtccttaagttgtccagtccactttccattcccaagaacgaagaagactgttgggttgaatagcctctactcctggacagtacaggacccagatctgggaaagagagtagagttagggaaggagttagaccatgtgacattctcactctcttaagtttctttgtcttctgagaagaaagagccgcttttctcagtatttcaaacagaggtcagtctcaaacataagggtgagtaggcgatgggctggttctatgaaattctacaagtatttattgagcgcctgtgctgtgcccagtactgcccttggcaccactgagttacagaagaaataatacaacatgcgatctttgccctctgggagctcacagtttatttagggagataggctatacacaggaaacagtaacaagggctgccagctcccagccctccctcactccagctgttctctccagttcttccagatgtcctccaggtgtctgctgcccccccagggccccgaggccgccaggccccaacctcacccccacgcatgacggtgcatgaggggcaggagctggcactgggctgcctggcgaggacaagcacacagaagcacacacacctggcagtgtcctttgggcgatctgtgcccgaggcaccagttgggcggtcaactctgcaggaagtggtgggaatccggtcagacttggccgtggaggctggagctccctatgctgagcgattggctgcaggggagcttcgtctgggcaaggaagggaccgatcggtaccgcatggtagtagggggtgcccaggcaggggacgcaggcacctaccactgcactgccgctgagtggattcaggatcctgatggcagctgggcccagattgcagagaaaagggccgtcctggcccacgtggatgtgcagacgctgtccagccagctggcagtgacagtggggcctggtgaacgtcggatcggcccaggggagcccttggaactgctgtgcaatgtgtcaggggcacttcccccagcaggccgtcatgctgcatactctgtaggttgggagatggcacctgcgggggcacctgggcccggccgcctggtagcccagctggacacagagggtgtgggcagcctgggccctggctatgagggccgacacattgccatggagaaggtggcatccagaacataccggctacggctagaggctgccaggcctggtgatgcgggcacctaccgctgcctcgccaaagcctatgttcgagggtctgggacccggcttcgtgaagcagccagtgcccgttcccggcctctccctgtacatgtgcgggaggaaggtgagagggggctgggccctggacggggttcgaggattgtcaaccccttttccttctgtttccatgacccctccctctccgtcagctgtttgcaggccacagcctcacgctcctcttctaccaagattgtgggaataccctgagtttcctggggcggtgcttggcggcagccctaaccccatgctccctggccctgcaccctctgaccccatgtcctttgttctgcaggtgtggtgctggaggctgtggcatggctagcaggaggcacagtgtaccgcggggagactgcctccctgctgtgcaacatctctgtgcggggtggccccccaggactgcggctggccgccagctggtgggtggagcgaccagaggacggagagctcagctctgtccctgcccagctggtgggtggcgtaggccaggatggtgtggcagagctgggagtccggcctggaggaggccctgtcagcgtagagctggtggggccccgaagccatcggctgagactacacagcttggggcccgaggatgaaggcgtgtaccactgtgcccccagcgcctgggtgcagcatgccgactacagctggtaccaggcgggcagtgcccgctcagggcctgttacagtctacccctacatgcatgccctggacaccctatttgtgcctctgctggtgggtacaggggtggccctagtcactggtgccactgtccttggtaccatcacttgctgcttcatgaagaggcttcgaaaacggtgatcccttactccccaggtcttgcaggtgtcgactgtcttccggcccagctccaagccctcctctggttgcctggacaccctctccctctgtccactcttcctttaatttatttgacctcccactacccagaatgggagacgtgcctccccttccccactccttccctcccaagcccctccctctggccttctgttcttgatctcttagggatcctatagggaggccatttcctgtcctggaattagtttttctaaaatgtgaataaacttgttttataaaaaaaaaaaaaaaaa

SEQ ID NO: 3 - DNA, 936 bp, Homo Sapiens
atgacggtgcatgaggggcaggagctggcactgggctgcctggcgaggacaagcacacagaagcacacacacctggcagtgtcctttgggcgatctgtgcccgaggcaccagttgggcggtcaactctgcaggaagtggtgggaatccggtcagacttggccgtggaggctggagctccctatgctgagcgattggctgcaggggagcttcgtctgggcaaggaagggaccgatcggtaccgcatggtagtagggggtgcccaggcaggggacgcaggcacctaccactgcactgccgctgagtggattcaggatcctgatggcagctgggcccagattgcagagaaaagggccgtcctggcccacgtggatgtgcagacgctgtccagccagctggcagtgacagtggggcctggtgaacgtcggatcggcccaggggagcccttggaactgctgtgcaatgtgtcaggggcacttcccccagcaggccgtcatgctgcatactctgtaggttgggagatggcacctgcgggggcacctgggcccggccgcctggtagcccagctggacacagagggtgtgggcagcctgggccctggctatgagggccgacacattgccatggagaaggtggcatccagaacataccggctacggctagaggctgccaggcctggtgatgcgggcacctaccgctgcctcgccaaagcctatgttcgagggtctgggacccggcttcgtgaagcagccagtgcccgttcccggcctctccctgtacatgtgcgggaggaaggtgagagggggctgggccctggacggggttcgaggattgtcaaccccttttccttctgtttccatgacccctccctctccgtcagctgtttgcaggccacagcctcacgctcctcttctaccaagattgtgggaataccctga

SEQ ID NO: 4 - protein, 311 aa, Homo sapiens [sequence omitted]

SEQ ID NO: 5 - DNA, 1842 bp, Homo sapiens
atgggcgccctcaggcccacgctgctgccgccttcgctgccgctgctgctgctgctaatgctaggaatgggatgctgggcccgggaggtgctggtccccgaggggcccttgtaccgcgtggctggcacagctgtctccatctcctgcaatgtgaccggctatgagggccctgcccagcagaacttcgagtggttcctgtataggcccgaggccccagatactgcactgggcattgtcagtaccaaggatacccagttctcctatgctgtcttcaagtcccgagtggtggcgggtgaggtgcaggtgcagcgcctacaaggtgatgccgtggtgctcaagattgcccgcctgcaggcccaggatgccggcatttatgagtgccacaccccctccactgatacccgctacctgggcagctacagcggcaaggtggagctgagagttcttccagatgtcctccaggtgtctgctgcccccccagggccccgaggccgccaggccccaacctcacccccacgcatgacggtgcatgaggggcaggagctggcactgggctgcctggcgaggacaagcacacagaagcacacacacctggcagtgtcctttgggcgatctgtgcccgaggcaccagttgggcggtcaactctgcaggaagtggtgggaatccggtcagacttggccgtggaggctggagctccctatgctgagcgattggctgcaggggagcttcgtctgggcaaggaagggaccgatcggtaccgcatggtagtagggggtgcccaggcaggggacgcaggcacctaccactgcactgccgctgagtggattcaggatcctgatggcagctgggcccagattgcagagaaaagggccgtcctggcccacgtggatgtgcagacgctgtccagccagctggcagtgacagtggggcctggtgaacgtcggatcggcccaggggagcccttggaactgctgtgcaatgtgtcaggggcacttcccccagcaggccgtcatgctgcatactctgtaggttgggagatggcacctgcgggggcacctgggcccggccgcctggtagcccagctggacacagagggtgtgggcagcctgggccctggctatgagggccgacacattgccatggagaaggtggcatccagaacataccggctacggctagaggctgccaggcctggtgatgcgggcacctaccgctgcctcgccaaagcctatgttcgagggtctgggacccggcttcgtgaagcagccagtgcccgttcccggcctctccctgtacacgtgcgggaggaaggtgtggtgctggaagctgtggcatggctagcaggaggcacagtgtaccgcggggagactgcctccctgctgtgcaacatctctgtgcggggtggccccccaggactgcggctggccgccagctggtgggtggagcgaccagaggacggagagctcagctctgtccctgcccagctggtgggtggcgtaggccaggatggtgtggcagagctgggagtccggcctggaggaggccctgtcagcgtagagctggtggggccccgaagccatcggctgagactacacagcttggggcccgaggatgaaggcgtgtaccactgtgcccccagcgcctgggtgcagcatgccgactacagctggtaccaggcgggcagtgcccgctcagggcctgttacagtctacccctacatgcatgccctggacaccctatttgtgcctctgctggtgggtacaggggtggccctagtcactggtgccactgtccttggtaccatcacttgctgcttcatgaagaggcttcgaaaacggtga

SEQ ID NO: 6 - protein, 613 aa, Homo sapiens
MGALRPTLLPPSLPLLLLLMLGMGCWAREVLVPEGPLYRVAGTAVSISCNVTGYEGPAQQNFEWFLYRPEAPDTALGIVSTKDTQFSYAVFKSRVVAGEVQVQRLQGDAVVLKIARLQAQDAGIYECHTPSTDTRYLGSYSGKVELRVLPDVLQVSAAPPGPRGRQAPTSPPRMTVHEGQELALGCLARTSTQKHTHLAVSFGRSVPEAPVGRSTLQEVVGIRSDLAVEAGAPYAERLAAGELRLGKEGTDRYRMVVGGAQAGDAGTYHCTAAEWIQDPDGSWAQIAEKRAVLAHVDVQTLSSQLAVTVGPGERRIGPGEPLELLCNVSGALPPAGRHAAYSVGWEMAPAGAPGPGRLVAQLDTEGVGSLGPGYEGRHIAMEKVASRTYRLRLEAARPGDAGTYRCLAKAYVRGSGTRLREAASARSRPLPVHVREEGVVLEAVAWLAGGTVYRGETASLLCNISVRGGPPGLRLAASWWVERPEDGELSSVPAQLVGGVGQDGVAELGVRPGGGPVSVELVGPRSHRLRLHSLGPEDEGVYHCAPSAWVQHADYSWYQAGSARSGPVTVYPYMHALDTLFVPLLVGTGVALVTGATVLGTITCCFMKRLRKR

SEQ ID NO: 7 - DNA, 4735 bp, Homo sapiens
cggccccggcgccatgagtggcggcggcggcggagggggctcggcgcccagtcgcttcgccgactactttgtcatctgcggactggacacggagaccgggctggagccggacgagctgtcggcattatgccagtacatacaggcttctaaagccagggatggtgccagccctttcatttcaagtacgactgaaggagaaaattttgagcagacaccattgagaagaacattcaaatctaaggtccttgcacgatatcctgagaacgtagaatggaatccctttgaccaagatgcagtaggaatgctatgtatgccgaaagggctggcattcaagacccaggctgatcccagggagccccaattccatgcctttattatcacaagggaggatggctctcggacatttgggtttgccctcacattttatgaagaggtgactagcaagcagatctgcagtgcaatgcagaccctctaccacatgcacaatgctgagtatgatgtcctacatgctccccctgctgatgacagagaccagagcagcatggaggatggtgaagacactcctgtgaccaaactgcagcgcttcaactcctatgacattagccgggacactctctacgtctctaagtgcatctgcctcatcacacccatgtctttcatgaaggcatgtcggagcgtgctggagcaactccaccaggcagtcacttcacctcagccccctccactgccccttgagagctacatatacaacgtactctacgaggtgccgctcccacctcctggccggtccttgaagttttctggggtctatgggccaataatctgccagagaccaagtaccaatgagcttcccctatttgactttcctgtcaaagaggtttttgaactgctcggggtggagaatgtgtttcagctttttacttgtgcccttctggagtttcaaatcctgctctactcacagcattaccagagactgatgactgtggcggagacgattacagctctcatgtttcctttccagtggcagcatgtctatgtccctattctcccagcttctctcctgcatttcttagatgctcctgttccatacctgatgggtttgcattccaatggcctggatgaccggtcaaagctggagctgcctcaagaggctaacctctgctttgtggacattgacaaccacttcattgagttgccagaggacttgccacagttccccaacaaattggagtttgtccaggaagtctctgagattctcatggcatttggaattccccctgaagggaatcttcattgcagtgagagtgcctccaagctgaagaggctgcgggcctctgagcttgtctcggacaagaggaatgggaacattgctggctcccctttgcattcctacgagcttcttaaggagaatgaaactattgcccggctgcaagccttggtcaagagaactggggtgagcctggaaaagttggaagtgcgtgaagaccccagcagcaataaggatctcaaagttcagtgtgatgaagaagaactcaggatttaccagctaaacattcagatccgggaagtttttgcaaatcgtttcactcagatgtttgcagattatgaggtgtttgtcatccaacccagccaggataaggaatcctggtttaccaacagggagcaaatgcaaaactttgataaagcatcttttctgtcagatcagcctgagccctacctgcccttcctctcaagattcctggagacccagatgtttgcatctttcattgacaacaaaataatgtgtcatgatgatgatgataaagaccctgtactccgggtatttgattcccgagttgacaagatcaggctgttgaatgttcggacacctactctccgtacatccatgtaccagaagtgtaccactgtggatgaagcagagaaagcaattgagctgcgtctggcaaaaattgaccatactgcaattcacccacatttacttgacatgaagattggacaagggaaatatgagccgggcttcttccctaagctgcagtctgatgtactttccactgggccagccagcaacaagtggacgaaaaggaatgcccctgcccagtggaggcggaaagatcggcagaagcagcacacagaacacctgcgtttagataatgaccagagggagaagtacatccaggaagccaggactatgggcagcactatccgccagcccaaactgtccaacctctctccatcagtgattgcccagaccaattggaagtttgtagagggcctgctgaaggaatgccgcaataagaccaagaggatgctggtggaaaagatgggccgagaagctgtggagctagggcatggggaggtgaacatcacaggggtggaagagaacaccctgattgccagcctttgtgatctcctggaaaggatctggagtcatggactacaagtgaaacaggggaaatcagccttatggtcccacctgttacattatcaggacaaccggcagagaaaactcacatcaggaagcctcagtacctcaggaatacttcttgattcagaacgtaggaagtctgatgccagctcactcatgcctcccctgaggatctccctgattcaggatatgaggcacatccagaacatcggggaaatcaagactgatgtgggaaaggccagagcatgggtgcgactgtccatggaaaaaaagttactttccagacacctgaagcagctcctctcagaccatgagctcaccaaaaagttatataagcgctatgccttcctgcgctgtgatgacgagaaggagcagttcctctatcacctcctgtctttcaatgccgtcgattacttttgcttcaccaatgtcttcacaactatcctgatcccgtaccacattctgatcgtaccaagcaagaagctggggggctccatgttcactgccaacccatggatctgtatatcaggagaattgggtgagacacagatcatgcagattcccaggaatgtgctagagatgaccttcgagtgccagaacttggggaagcttactactgtccagattggccatgataactctgggctgtatgccaaatggctggtggagtatgtgatggtcaggaatgagatcacaggacatacctacaagttcccgtgtggccggtggttagggaagggcatggatgatggaagcctggagcggatcctagttggggagctgctcacatcccagcctgaggtggatgagaggccatgccggaccccgccgctgcagcagtcccccagtgtcatccggaggcttgttaccatctcacccaacaacaagcccaagctgaacactgggcagatccaggagtccatcggggaggcagtcaatggcattgtgaagcacttccataagcctgagaaagagcgaggcagtctgacgctgttgctctgtggagagtgtggccttgtctcggccttggaacaggctttccagcatggatttaaatcgccccggctcttcaaaaatgtcttcatttgggatttcctggaaaaagcacaaacctattatgagacattagagaagaatgaagtagtccctgaggaaaactggcatacaagagcccggaacttctgccgatttgtcactgcaatcaacaatactccccggaacatcggcaaggatggcaagtttcagatgctggtgtgcttgggagccagagatcacctcctacaccactggattgccctgctggctgactgccccatcactgcacacatgtatgaggatgtggcactgatcaaagaccatacacttgtcaattccttgattcgtgtgctgcagacattgcaggagttcaacatcacgctggagacgtcccttgtcaagggcatcgacatctgacctcccagcaccagccagcagcaggactgagaaagactcaccctgcagctctgaccttttttcccaaagggacttaagcgattgtgcaggagtaggagacaaaatgtacactcactgtaaaaagaaaactagaggatttttggaataaataatctattttagagtttatttgctgatttgctttttacacactttcatgtgaaagagtgatagggagagggagcgaggctggtgccgcttattttgaagctggtgccctccctcgccgtggccacatgctggaagcctgaggcctccctggactgagcctgtggcactgcgtgcgggacagttatgtttccttgccccgtcgcattaatgaggcccttccacatcatttttaaactaatgtttttctatattaacattattatggatatttggctttcataggccacacacaggtgtgctgcgcgggaagccccatgctccaatcaaagggatttttagtagtgcctctaagcaagcaccgatgagtcagtcccacgtattttcttttttgtcagtattgtttgggaaggagacatgccgggatgtgtcatcgtgccaaataccacatttcctgttggcacagtttcacagaagtaaacataagcatgttttaacaggtttttcttttcttttttcttttttaaaatgttttatttatttaacccgccattgtgtgtttttaagtattttctttttttaaggaaaggaaaagcttgtcacaatctaactggctatgttattattattaaatttatgttttgcaacttagaaaccagctacagtatggcccacttaataaaacacctgaaacaaaaaaaaaaaaaaaaa

SEQ ID NO: 8 - DNA, 3864 bp, Homo sapiens
atgagtggcggcggcggcggagggggctcggcgcccagtcgcttcgccgactactttgtcatctgcggactggacacggagaccgggctggagccggacgagctgtcggcattatgccagtacatacaggcttctaaagccagggatggtgccagccctttcatttcaagtacgactgaaggagaaaattttgagcagacaccattgagaagaacattcaaatctaaggtccttgcacgatatcctgagaacgtagaatggaatccctttgaccaagatgcagtaggaatgctatgtatgccgaaagggctggcattcaagacccaggctgatcccagggagccccaattccatgcctttattatcacaagggaggatggctctcggacatttgggtttgccctcacattttatgaagaggtgactagcaagcagatctgcagtgcaatgcagaccctctaccacatgcacaatgctgagtatgatgtcctacatgctccccctgctgatgacagagaccagagcagcatggaggatggtgaagacactcctgtgaccaaactgcagcgcttcaactcctatgacattagccgggacactctctacgtctctaagtgcatctgcctcatcacacccatgtctttcatgaaggcatgtcggagcgtgctggagcaactccaccaggcagtcacttcacctcagccccctccactgccccttgagagctacatatacaacgtactctacgaggtgccgctcccacctcctggccggtccttgaagttttctggggtctatgggccaataatctgccagagaccaagtaccaatgagcttcccctatttgactttcctgtcaaagaggtttttgaactgctcggggtggagaatgtgtttcagctttttacttgtgcccttctggagtttcaaatcctgctctactcacagcattaccagagactgatgactgtggcggagacgattacagctctcatgtttcctttccagtggcagcatgtctatgtccctattctcccagcttctctcctgcatttcttagatgctcctgttccatacctgatgggtttgcattccaatggcctggatgaccggtcaaagctggagctgcctcaagaggctaacctctgctttgtggacattgacaaccacttcattgagttgccagaggacttgccacagttccccaacaaattggagtttgtccaggaagtctctgagattctcatggcatttggaattccccctgaagggaatcttcattgcagtgagagtgcctccaagctgaagaggctgcgggcctctgagcttgtctcggacaagaggaatgggaacattgctggctcccctttgcattcctacgagcttcttaaggagaatgaaactattgcccggctgcaagccttggtcaagagaactggggtgagcctggaaaagttggaagtgcgtgaagaccccagcagcaataaggatctcaaagttcagtgtgatgaagaagaactcaggatttaccagctaaacattcagatccgggaagtttttgcaaatcgtttcactcagatgtttgcagattatgaggtgtttgtcatccaacccagccaggataaggaatcctggtttaccaacagggagcaaatgcaaaactttgataaagcatcttttctgtcagatcagcctgagccctacctgcccttcctctcaagattcctggagacccagatgtttgcatctttcattgacaacaaaataatgtgtcatgatgatgatgataaagaccctgtactccgggtatttgattcccgagttgacaagatcaggctgttgaatgttcggacacctactctccgtacatccatgtaccagaagtgtaccactgtggatgaagcagagaaagcaattgagctgcgtctggcaaaaattgaccatactgcaattcacccacatttacttgacatgaagattggacaagggaaatatgagccgggcttcttccctaagctgcagtctgatgtactttccactgggccagccagcaacaagtggacgaaaaggaatgcccctgcccagtggaggcggaaagatcggcagaagcagcacacagaacacctgcgtttagataatgaccagagggagaagtacatccaggaagccaggactatgggcagcactatccgccagcccaaactgtccaacctctctccatcagtgattgcccagaccaattggaagtttgtagagggcctgctgaaggaatgccgcaataagaccaagaggatgctggtggaaaagatgggccgagaagctgtggagctagggcatggggaggtgaacatcacaggggtggaagagaacaccctgattgccagcctttgtgatctcctggaaaggatctggagtcatggactacaagtgaaacaggggaaatcagccttatggtcccacctgttacattatcaggacaaccggcagagaaaactcacatcaggaagcctcagtacctcaggaatacttcttgattcagaacgtaggaagtctgatgccagctcactcatgcctcccctgaggatctccctgattcaggatatgaggcacatccagaacatcggggaaatcaagactgatgtgggaaaggccagagcatgggtgcgactgtccatggaaaaaaagttactttccagacacctgaagcagctcctctcagaccatgagctcaccaaaaagttatataagcgctatgccttcctgcgctgtgatgacgagaaggagcagttcctctatcacctcctgtctttcaatgccgtcgattacttttgcttcaccaatgtcttcacaactatcctgatcccgtaccacattctgatcgtaccaagcaagaagctggggggctccatgttcactgccaacccatggatctgtatatcaggagaattgggtgagacacagatcatgcagattcccaggaatgtgctagagatgaccttcgagtgccagaacttggggaagcttactactgtccagattggccatgataactctgggctgtatgccaaatggctggtggagtatgtgatggtcaggaatgagatcacaggacatacctacaagttcccgtgtggccggtggttagggaagggcatggatgatggaagcctggagcggatcctagttggggagctgctcacatcccagcctgaggtggatgagaggccatgccggaccccgccgctgcagcagtcccccagtgtcatccggaggcttgttaccatctcacccaacaacaagcccaagctgaacactgggcagatccaggagtccatcggggaggcagtcaatggcattgtgaagcacttccataagcctgagaaagagcgaggcagtctgacgctgttgctctgtggagagtgtggccttgtctcggccttggaacaggctttccagcatggatttaaatcgccccggctcttcaaaaatgtcttcatttgggatttcctggaaaaagcacaaacctattatgagacattagagaagaatgaagtagtccctgaggaaaactggcatacaagagcccggaacttctgccgatttgtcactgcaatcaacaatactccccggaacatcggcaaggatggcaagtttcagatgctggtgtgcttgggagccagagatcacctcctacaccactggattgccctgctggctgactgccccatcactgcacacatgtatgaggatgtggcactgatcaaagaccatacacttgtcaattccttgattcgtgtgctgcagacattgcaggagttcaacatcacgctggagacgtcccttgtcaagggcatcgacatctga

SEQ ID NO: 9 - protein, 1287 aa, Homo sapiens
MSGGGGGGGSAPSRFADYFVICGLDTETGLEPDELSALCQYIQASKARDGASPFISSTTEGENFEQTPLRRTFKSKVLARYPENVEWNPFDQDAVGMLCMPKGLAFKTQADPREPQFHAFIITREDGSRTFGFALTFYEEVTSKQICSAMQTLYHMHNAEYDVLHAPPADDRDQSSMEDGEDTPVTKLQRFNSYDISRDTLYVSKCICLITPMSFMKACRSVLEQLHQAVTSPQPPPLPLESYIYNVLYEVPLPPPGRSLKFSGVYGPIICQRPSTNELPLFDFPVKEVFELLGVENVFQLFTCALLEFQILLYSQHYQRLMTVAETITALMFPFQWQHVYVPILPASLLHFLDAPVPYLMGLHSNGLDDRSKLELPQEANLCFVDIDNHFIELPEDLPQFPNKLEFVQEVSEILMAFGIPPEGNLHCSESASKLKRLRASELVSDKRNGNIAGSPLHSYELLKENETIARLQALVKRTGVSLEKLEVREDPSSNKDLKVQCDEEELRIYQLNIQIREVFANRFTQMFADYEVFVIQPSQDKESWFTNREQMQNFDKASFLSDQPEPYLPFLSRFLETQMFASFIDNKIMCHDDDDKDPVLRVFDSRVDKIRLLNVRTPTLRTSMYQKCTTVDEAEKAIELRLAKIDHTAIHPHLLDMKIGQGKYEPGFFPKLQSDVLSTGPASNKWTKRNAPAQWRRKDRQKQHTEHLRLDNDQREKYIQEARTMGSTIRQPKLSNLSPSVIAQTNWKFVEGLLKECRNKTKRMLVEKMGREAVELGHGEVNITGVEENTLIASLCDLLERIWSHGLQVKQGKSALWSHLLHYQDNRQRKLTSGSLSTSGILLDSERRKSDASSLMPPLRISLIQDMRHIQNIGEIKTDVGKARAWVRLSMEKKLLSRHLKQLLSDHELTKKLYKRYAFLRCDDEKEQFLYHLLSFNAVDYFCFTNVFTTILIPYHILIVPSKKLGGSMFTANPWICISGELGETQIMQIPRNVLEMTFECQNLGKLTTVQIGHDNSGLYAKWLVEYVMVRNEITGHTYKFPCGRWLGKGMDDGSLERILVGELLTSQPEVDERPCRTPPLQQSPSVIRRLVTISPNNKPKLNTGQIQESIGEAVNGIVKHFHKPEKERGSLTLLLCGECGLVSALEQAFQHGFKSPRLFKNVFIWDFLEKAQTYYETLEKNEVVPEENWHTRARNFCRFVTAINNTPRNIGKDGKFQMLVCLGARDHLLHHWIALLADCPITAHMYEDVALIKDHTLVNSLIRVLQTLQEFNITLETSLVKGIDI

SEQ ID NO: 10 - DNA, 3358 bp, Homo sapiens
gtctggctaactcatctccagacctaagttgggaaaggggaggggagcgcggaaagcggagaagccggaggagacgctccggagtgaccagcgacggctgaacgcagcccgccctccccaagtcgacgccgcaacccggggaaagcaggaggaccatgcggcagtagcagccatgctgccctttctgctggccacactgggcaccacagccctcaacaacagcaaccccaaggactactgctacagcgcccgcatccgcagcactgtcctgcagggcctgccctttgggggcgtccccaccgtgctggctctcgacttcatgtgcttccttgcactgctgttcttattctctatcctccggaaggtggcctgggactatgggcggctggccttggtgacagatgcagacaggcttcggcggcaggagagggaccgagtggaacaggaatatgtggcttcagctatgcacggggacagccatgaccggtatgagcgtctcacctctgtctccagctccgttgactttgaccaaagggacaatggtttctgttcctggctgacagccatcttcaggataaaggatgatgagatccgggacaaatgtgggggcgatgccgtgcactacctgtcctttcagcggcacatcatcgggctgctggtggttgtgggcgtcctctccgtaggcatcgtgctgcctgtcaacttctcaggggacctgctggagaacaatgcctacagctttgggagaaccaccattgccaacttgaaatcagggaacaacctgctatggctgcacacctccttcgccttcctgtatctgctgctcaccgtctacagcatgcgtagacacacctccaagatgcgctacaaggaggatgatctggtgaagcggaccctcttcatcaatggaatctccaaatatgcagagtcagaaaagatcaagaagcattttgaggaagcctaccccaactgcacagttctcgaagcccgcccgtgttacaacgtggctcgcctaatgttcctcgatgcagagaggaagaaggccgagcggggaaagctgtacttcacaaacctccagagcaaggagaacgtgcctaccatgatcaaccccaagccctgtggccacctctgctgctgtgtggtgcgaggctgtgagcaggtggaggccattgagtactacacaaagctggagcagaagctgaaggaagactacaagcgggagaaggagaaggtgaatgagaagcctcttggcatggcctttgtcaccttccacaatgagactatcaccgccatcatcctgaaggacttcaacgtgtgtaaatgccagggctgcacctgccgtggggagccacgcccctcatcctgcagcgagtccctgcacatctccaactggaccgtgtcctatgcccctgaccctcagaacatctactgggagcacctctccatccgaggcttcatctggtggctgcgctgcctggtcatcaatgtcgtcctcttcatcctcctcttcttcctcaccactccagccatcatcatcaccaccatggacaagttcaacgtcaccaagcctgtggagtacctcaacaaccccatcatcacccagttcttccccaccctgctgctgtggtgcttctcggccctccttcccaccatcgtctactactcagccttctttgaagcccactggacacgctctggggagaacaggacaaccatgcacaagtgctacactttcctcatcttcatggtgctgctcctaccctcgctgggactgagcagcctggacctcttcttccgctggctctttgataagaaattcttggctgaggcagctattcggtttgagtgtgtgttcctgcccgacaacggcgccttcttcgtgaactacgtcattgcctcagcctttatcggcaacgccatggacctgctgcgcatcccaggcctgctcatgtacatgatccggctctgcctggcgcgctcggccgccgagaggcgcaacgtgaagcggcatcaggcctacgagttccagtttggcgcagcctacgcctggatgatgtgcgtcttcacggtggtcatgacctacagtatcacctgccccatcatcgtgcccttcgggctcatgtacatgctgctgaagcacctggtagacaggtacaatctctactacgcctacctgccggccaagctggacaagaagatccactcgggggctgtgaaccaggtggtggccgcgcccatcctctgcctcttctggctgctcttcttttccaccatgcgcacggggttcctagctcccacgtctatgttcacatttgtggtcctggtcatcaccatcgtcatctgtctctgccacgtctgctttggacacttcaaatacctcagtgcccacaactacaagattgagcacacggagacagatactgtggaccccagaagcaatggacggccccccactgctgctgctgtccccaaatctgcgaaatacatcgctcaggtgctgcaggactcagaggtggacggggatggggatggggctcctgggagctcaggggatgagcccccatcatcctcatcccaagatgaggagttgctgatgccacccgacgccctcacggacacagacttccagtcttgcgaggacagcctcatagagaatgagattcaccagtaaggggagggaggggccctggaggccacatcctgccccaccccacccccactcccacggacactaaaacgctaataatttattagatctaaagccccttcctccccagcccctgctttcattaaggtatttaaacttgggggtttcactgctctcccccatgatggagggagggagccccccaacctcagtgaggagagccccgagccggccccggggcaaagaggggtgcagagggagttcccccagatcagtaccccccacccctccccagctagtagcatgaccaggagagggttaatgagagccaagaggagtacctggtgcacctggtgccggtggctggagacctggggggcaggtggatctggggctgttcccccccctccgttttttccaccccacagttcctcctgggatctggccctccagggaagtggagcctccagcccctaggggatgcatgaggggggagggggtgctgagtgggaggaagagtcaggctcacagctggggtggcctgggggtgggggtgggcaaggctgacactggaaaatgggtttttgcactgtttttttttttggtttttttgttcttttttgtttttttcctttaaaataaaaacaaagaaaagctctgaaaaaaaaaaaaaaaaaaaaaaaaaaaaaaaaaaaaaaaaaaaaaaaaaaa

SEQ ID NO: 11 - DNA, 2499 bp, Homo sapiens
atgctgccctttctgctggccacactgggcaccacagccctcaacaacagcaaccccaaggactactgctacagcgcccgcatccgcagcactgtcctgcagggcctgccctttgggggcgtccccaccgtgctggctctcgacttcatgtgcttccttgcactgctgttcttattctctatcctccggaaggtggcctgggactatgggcggctggccttggtgacagatgcagacaggcttcggcggcaggagagggaccgagtggaacaggaatatgtggcttcagctatgcacggggacagccatgaccggtatgagcgtctcacctctgtctccagctccgttgactttgaccaaagggacaatggtttctgttcctggctgacagccatcttcaggataaaggatgatgagatccgggacaaatgtgggggcgatgccgtgcactacctgtcctttcagcggcacatcatcgggctgctggtggttgtgggcgtcctctccgtaggcatcgtgctgcctgtcaacttctcaggggacctgctggagaacaatgcctacagctttgggagaaccaccattgccaacttgaaatcagggaacaacctgctatggctgcacacctccttcgccttcctgtatctgctgctcaccgtctacagcatgcgtagacacacctccaagatgcgctacaaggaggatgatctggtgaagcggaccctcttcatcaatggaatctccaaatatgcagagtcagaaaagatcaagaagcattttgaggaagcctaccccaactgcacagttctcgaagcccgcccgtgttacaacgtggctcgcctaatgttcctcgatgcagagaggaagaaggccgagcggggaaagctgtacttcacaaacctccagagcaaggagaacgtgcctaccatgatcaaccccaagccctgtggccacctctgctgctgtgtggtgcgaggctgtgagcaggtggaggccattgagtactacacaaagctggagcagaagctgaaggaagactacaagcgggagaaggagaaggtgaatgagaagcctcttggcatggcctttgtcaccttccacaatgagactatcaccgccatcatcctgaaggacttcaacgtgtgtaaatgccagggctgcacctgccgtggggagccacgcccctcatcctgcagcgagtccctgcacatctccaactggaccgtgtcctatgcccctgaccctcagaacatctactgggagcacctctccatccgaggcttcatctggtggctgcgctgcctggtcatcaatgtcgtcctcttcatcctcctcttcttcctcaccactccagccatcatcatcaccaccatggacaagttcaacgtcaccaagcctgtggagtacctcaacaaccccatcatcacccagttcttccccaccctgctgctgtggtgcttctcggccctccttcccaccatcgtctactactcagccttctttgaagcccactggacacgctctggggagaacaggacaaccatgcacaagtgctacactttcctcatcttcatggtgctgctcctaccctcgctgggactgagcagcctggacctcttcttccgctggctctttgataagaaattcttggctgaggcagctattcggtttgagtgtgtgttcctgcccgacaacggcgccttcttcgtgaactacgtcattgcctcagcctttatcggcaacgccatggacctgctgcgcatcccaggcctgctcatgtacatgatccggctctgcctggcgcgctcggccgccgagaggcgcaacgtgaagcggcatcaggcctacgagttccagtttggcgcagcctacgcctggatgatgtgcgtcttcacggtggtcatgacctacagtatcacctgccccatcatcgtgcccttcgggctcatgtacatgctgctgaagcacctggtagacaggtacaatctctactacgcctacctgccggccaagctggacaagaagatccactcgggggctgtgaaccaggtggtggccgcgcccatcctctgcctcttctggctgctcttcttttccaccatgcgcacggggttcctagctcccacgtctatgttcacatttgtggtcctggtcatcaccatcgtcatctgtctctgccacgtctgctttggacacttcaaatacctcagtgcccacaactacaagattgagcacacggagacagatactgtggaccccagaagcaatggacggccccccactgctgctgctgtccccaaatctgcgaaatacatcgctcaggtgctgcaggactcagaggtggacggggatggggatggggctcctgggagctcaggggatgagcccccatcatcctcatcccaagatgaggagttgctgatgccacccgacgccctcacggacacagacttccagtcttgcgaggacagcctcatagagaatgagattcaccagtaa

SEQ ID NO: 12 - protein, 832 aa, Homo sapiens
MLPFLLATLGTTALNNSNPKDYCYSARIRSTVLQGLPFGGVPTVLALDFMCFLALLFLFSILRKVAWDYGRLALVTDADRLRRQERDRVEQEYVASAMHGDSHDRYERLTSVSSSVDFDQRDNGFCSWLTAIFRIKDDEIRDKCGGDAVHYLSFQRHIIGLLVVVGVLSVGIVLPVNFSGDLLENNAYSFGRTTIANLKSGNNLLWLHTSFAFLYLLLTVYSMRRHTSKMRYKEDDLVKRTLFINGISKYAESEKIKKHFEEAYPNCTVLEARPCYNVARLMFLDAERKKAERGKLYFTNLQSKENVPTMINPKPCGHLCCCVVRGCEQVEAIEYYTKLEQKLKEDYKREKEKVNEKPLGMAFVTFHNETITAIILKDFNVCKCQGCTCRGEPRPSSCSESLHISNWTVSYAPDPQNIYWEHLSIRGFIWWLRCLVINVVLFILLFFLTTPAIIITTMDKFNVTKPVEYLNNPIITQFFPTLLLWCFSALLPTIVYYSAFFEAHWTRSGENRTTMHKCYTFLIFMVLLLPSLGLSSLDLFFRWLFDKKFLAEAAIRFECVFLPDNGAFFVNYVIASAFIGNAMDLLRIPGLLMYMIRLCLARSAAERRNVKRHQAYEFQFGAAYAWMMCVFTVVMTYSITCPIIVPFGLMYMLLKHLVDRYNLYYAYLPAKLDKKIHSGAVNQVVAAPILCLFWLLFFSTMRTGFLAPTSMFTFVVLVITIVICLCHVCFGHFKYLSAHNYKIEHTETDTVDPRSNGRPPTAAAVPKSAKYIAQVLQDSEVDGDGDGAPGSSGDEPPSSSSQDEELLMPPDALTDTDFQSCEDSLIENEIHQ

(19) United States
(12) Patent Application Publication
(10) Pub. No.: US 2005/0079572 A1
(43) Pub. Date: Apr. 14, 2005

(54) Organic compounds

(76) Inventors: Adelheid Cerwenka, Heidelberg (DE); Frank Stephan Kalthoff, Guntramsdorf (AT); William F Phares, Basel (CH)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 10/483,915

(22) PCT Filed: Jul. 16, 2002
(86) PCT No.: PCT/EP02/07924

Related U.S. Application Data
(60) Provisional application No. 60/305,932, filed on Jul. 17, 2001.
(60) Provisional application No. 60/313,550, filed on Aug. 20, 2001.
(60) Provisional application No. 60/315,123, filed on Aug. 27, 2001.
(60) Provisional application No. 60/318,484, filed on Sep. 10, 2001.

(51) Int. Cl. C12Q 1/68; C07H 21/04; C07K 14/74; C12N 5/08

(52) U.S. Cl. 435/69.1; 435/6; 435/320.1; 435/372; 530/350; 536/23.5

(57) ABSTRACT

An isolated BSAP1, GID 2.1, GID 2.2, GID3 and GID4 gene expressed in dendritic cells of the immune system, BASP1, GID 2.1, GID 2.2, GID3 and GID4 polypeptides expressed by such gene and their function in the identification of compo8unds which are (ant)agonists to BASP1, GID 2.1, GID 2.2, GID3 and GID4 polypeptides.

The present invention relates to genes isolated from dendritic cells and proteins (polypeptides) encoded by such genes, e.g. including the therapeutic intervention in diseases e.g. associated with abnormal immunological reactivity, by inhibiting or activating (modulating) the action of such genes/proteins in dendritic cells.

Dendritic cells (DCs) are professional antigen-presenting leukocytes which play a central role in the induction of primary immune responses and tolerance. In the immature state DCs may reside in different tissues of the body where they are capable of capturing antigen from invading pathogens and responding to inflammatory molecules. Following antigen capture DCs mature into antigen-presenting cells and migrate into lymphoid organs to activate T cells (Banchereau, M. and R. M. Steinman, Nature 329 [1998]245-252). For example, Langerhans cells (LC), residing in the skin, have been shown to present a variety of antigens that may be generated in or penetrate into skin. In contact hypersensitivity, topical application of a reactive hapten may activate LC to migrate out of the epidermis into draining lymph nodes, where LC may present antigen to selected T cells. During the contact between LC and T-cells, LC may provide signals to the T-cell that induce T-cell proliferation and differentiation into effector cells. Depending on the type of T-cells and the kind of interacting molecules involved, cytotoxic, regulatory and helper T-cells may be formed. DC's have also been shown to engulf all kinds of apoptic cells and may therefore play a critical role in the maintenance of tolerance to self-antigens (Steinman, R. M and K. Inaba, J. Leukoc. Biol. 66 [1999]205-208). Diseases in which DCs, as potent initiating and regulatory cells of immune responses, play a causal or contributory role may be targets for DC-specific pharmaceutical intervention, such as in chronic inflammatory diseases, autoimmune diseases, transplant rejection crises, and including e.g. inflammatory skin diseases such as contact hypersensitivity and atopic dermatitis; and in diseases characterized by immune suppression or hyporeactivity, including e.g. AIDS and cancer.

DCs may be isolated from peripheral blood by negative selection (enrichment) i.e. selective removal of other cell types that are present or separation from these other cell types like monocytes (CD14+), T-cells (CD3+), B-cells (CD19+) and NK-cells (CD16+) by capturing on specific monoclonal antibody (mAB)-coated magnetic beads or plastic surfaces, e.g. monocytes or immature progenitor cells by cultivation of cell isolated by capture with specific mAB as described, e.g. according to a method as conventional.

RNA may be isolated from DCs, cDNA and cDNA libraries may be generated; and differential gene expression patterns of DCs may be obtained by e.g. oligonucleotide fingerprinting, RNA-cDNA hybridization profiling, subtractive hybridization, or sequencing, e.g. according to methods as conventional.

We have found from cDNA synthesized from RNA of isolated human DCs, recombinant clones containing copies of genes having the names as set out in TABLE 1.

TABLE 1

Sequence of

| Gene name | nucleotides | amino acids |
|---|---|---|
| BASP1 | SEQ ID NO:1 | |
| GID2.1 | SEQ ID NO:2 | |
| GID2.1 CODING | SEQ ID NO:3 | SEQ ID NO:4 |
| GID2.2 CODING | SEQ ID NO:5 | SEQ ID NO:6 |
| GID3 | SEQ ID NO:7 | |
| GID3 CODING | SEQ ID NO:8 | SEQ ID NO:9 |
| GID4 | SEQ ID NO:10 | |
| GID4 CODING | SEQ ID NO:11 | SEQ ID NO:12 |

From cDNA libraries of isolated DCs, recombinant clones containing copies of the BASP1 (also named NAP22, or CAP) transcript may be identified, which have the nucleotide sequence SEQ ID NO:1, encoding the polypeptide identical to that in the SwissProt sequence database under the accession number P80723 for BASP1, that was derived from protein sequencing data (Mosevitsky, M. I., Biochimie 79 [1997]373-384). SEQ ID NO:1 differs by several substitutions, small deletions and insertions, to the polynucleotide sequences in GenBank, accession numbers AF039656 (NAP22)(Park, S., Mol. Cells 8 [1998]471-477) and NM_{—}006317 (7 nucleotide differences) and BC000518 (one nucleotide difference). The BASP1 protein is known to be expressed in spinal cord and brain (Iino, S. and S. Maekawa, Brain Res. 834 [1999] 66-73; Iino, S., Neuroscience 91 [1999] 1435-1444), where it has been observed to be localized on membrane microdomains (rafts) in neuronal cells, and this localization is cholesterol-dependent (Maekawa, S., J. Biol. Chem. 274 [1999] 21369-21374). It has been demonstrated that the BASP1 protein contains a calmodulin-binding domain (Takasaki, A., J. Biol. Chem. 274 [1999] 11848-11853), suggesting a functional role in metabolic activation in neuronal cells in response to calcium flux, and that changes in localization in neuronal cells occur during development of neuronal polarity (Kashihara, M., Neurosci. Res. 37 [2000]315-325).

We have now observed that BASP1 expression in DCs, with relevance to function of the gene in DCs and associated immunological reactivity, is induced in response to treatment with agents known to potentiate the ability of DCs to stimulate T cells, this property of DCs being central to their key regulatory role within the immune system. The induction of BASP1 gene expression in response to stimulation of DC is shown in FIG. 1. Furthermore, we have observed that the ectopic expression of BASP 1 can modulate expression of inflammatory cytokines, such as IL-8 (see Example 15 and FIG. 7).

We have also found that from cDNA synthesized from RNA of isolated human DC, recombinant clones containing copies of GID2.1 and GID2.2 transcripts of a GID2 gene (polynucleotide) may be isolated and identified. Re-amplification of the identified sequences directly from cDNA from DC and analysis of multiple clones reveals that the consensus sequences comprise the polynucleotide sequence SEQ ID NO:2, the polypeptide-coding sequence SEQ ID NO:3, and the correspondingly encoded polypeptide sequence SEQ ID NO:4 of the GID2.1 transcript; and the polynucleotide sequence SEQ ID NO:5 and the correspondingly encoded polypeptide sequence SEQ ID NO:6 of the GID2.2 transcript. SEQ ID NO:2 and SEQ ID NO:5 are presumably differentially spliced transcripts of the GID2 gene (polynucleotide), that reflects diversity in the coding potential of the GID2 gene. The encoded GID2 polypeptide sequences GID2.1 SEQ ID NO:4 and GID2.2 SEQ ID NO:6 are homologous to two known proteins: the V7 (CD101) leukocyte surface glycoprotein, and a regulatory protein of the protaglandin F2α receptor (FPRP); and to an uncharacterized KIAA1436 protein. V7 is expressed on activated T cells, monocytes, and granulocytes, as well as subpopulations of T cells and certain accessory cells, and has been shown to play a role in signal-1 transduction in T cells, i.e. in mediation of signals generated through crosslinking of the T cell receptor complex (Ruegg, C. L. et al., J. Immunol. 154 [1995] 4434-4443). Murine FPRP has been shown to bind physically to F2α receptor molecules, preventing their surface translocation and thus negatively regulating cellular stimulation by prostaglandin F2 (Orlicky, D. J. and S. K. Nordeen, Prostaglandins Leukot. Essent. Fatty Acids 55 [1996] 261-268; Orlicky D. J., Prostaglandins Leukot. Essent. Fatty Acids 54 [1996] 247-259). The GID2 polypeptides GID2.1 SEQ ID NO:4 and GID2.2 SEQ ID NO:6, as well as the homologous V7 protein and FPRP, contain multiple immunoglobulin-like and major histocompatibility complex domains, and are putative novel members of the immunoglobulin superfamily that is involved in critical intracellular signals necessary for many immune-related activities. We have observed that GID2 expression in DC, with relevance to function of the gene in DC and associated immunological reactivity, is induced in response to treatment with agents known to potentiate the ability of DC to stimulate T cells, this property of DC being central to their key regulatory role within the immune system. The induction of GID2 gene polynucleotide) expression in response to simulation of DC is shown in FIG. 2. Furthermore, we have observed that ectopic expression of GID2.2 in antigen-presenting cells positively regulates T cell proliferation (see Example 16 and FIGS. 8 and 9).

GID2 genes (polynucleotides) and polypeptides described herein are GID2 genes (polynucleotides) and polypeptides provided by the present invention.

We have further found that from cDNA synthesized from RNA of isolated human DC, recombinant clones containing copies of the transcripts of a GID3 gene may be isolated and identified. Re-amplification of the identified sequences directly from cDNA from DC, and analysis of multiple clones reveals that the consensus sequences comprise the polynucleotide sequences SEQ ID NO:7, the polypeptide-coding sequences SEQ ID NO:8, and the corresponding encoded polypeptide sequence SEQ ID NO:9 of the GID3 transcript. The latter sequence contains four potential N-glycosylation sites, specific to the consensus sequence Asn-Xaa-Ser/Thr (Marshall, R. D., Annu. Rev. Biochem. 41 [1972] 673-702) at positions 466, 760, 783 and 1274; and strong homology (4.7e-44) to the DENN (AEX-3) domain from aa residues 248-390. The human serine- and leucine-rich DENN protein possesses a RGD cellular adhesion motif and a leucine-zipper-like motif associated with protein dimerization, and shows partial homology to the receptor binding domain of tumor necrosis factor alpha. DENN is virtually identical to MADD, a human MAP kinase-activating death domain protein that interacts with type I tumor necrosis factor receptor. DENN displays significant homology to Rab3 GEP, a rat GDP/GTP exchange protein specific for Rab3 small G proteins implicated in intracellular vesicle trafficking. DENN also exhibits strong similarity to Caenorhabditis elegans AEX-3, which interacts with Rab3 to regulate synaptic vesicle release (Lim, D., et al., Genome 41 [1988] 543-552). Examples of other proteins containing DENN (AEX-3) domains are: a hypothetical protein from Schizosaccharomyces pombe (Q9Y7Q7), the human DENN protein (Q15741), the human MADD protein (O15293), the Caenorhabditis elegans protein AEX-3 (O02626), and the rat Rab3 small G protein (O08873). From homology relationships of the GID3 protein to orthologues, it may be deduced that GID3 may play an effector regulatory role as e.g. an adapter molecule in certain cellular processes that are GTP-dependent. The GID3 polypeptide sequences SEQ ID NO:9 are identical to the hypothetical human 147 KDA protein (TREMBL:Q9UFV0) and the KIAA1090 protein (one point mutational difference) (TREMBL:Q9UPR1); 95.8% identical (a 24-residue gap and numerous point mutations) to the hypothetical mouse 144.1 KDA protein (TREMBL:Q9QYZ2), containing the Rab6 GTP-binding protein associated protein fragment TREMBL:Q62146 identified in a yeast two-hybrid screen (Janoueix-Lerosey, I., et al., J. Biol. Chem 270 [1995] 14801-14808). The Rab proteins are Ras-like GTP-binding proteins involved in the regulation of vesicular transport through the endocytic and secretory pathway (Valencia, A., et al., Biochemistry 30 [1991] 4637-4648; Pfeffer, S. R., Curr. Opin. Cell Biol. 6 [1994] 522-526; Nuoffer, C. and W. E. Balch, Annu. Rev. Biochem. 63 [1994] 949-990), and Rab6 is thought to regulate intra-Golgi membrane transport (Goud, B., et al., Nature 345 [1990] 553-556; Martinez, O., et al. J. Cell Biol. 127 [1994] 1575-1588). The fragment of the mouse Rab6-associated homolog TREMBL:Q62146 was shown to preferentially interact with the GTP-bound form of Rab6 (Janoueix-Lerosey, I., et al., J. Biol. Chem 270 [1995] 14801-14808), and thus speculated to represent either an effector of Rab6 or a GAP protein. One other potential process in which GID3 may function is suggested by its relationship to the orthologous D. melanogaster Crag protein (23% identity/72.1% alignment) of the GenBank MF46398 sequence entry, identified as a calmodulin-binding protein (Xian-Zhong, S. X., et al. J. Biol Chem 274 [1998] 31297-31307). The latter physical interaction is thought to reflect a possible co-regulatory function with calmodulin in signal-transduced events, in which calmodulin is a major cellular sensor of calcium flux. In addition, calmodulin has been shown itself to bind to another Rab protein, Rab3A (Park, J. B., et al., J. Biol. Chem. 272 [1997] 20857-20865). We have observed that GID3 expression in DCs, with relevance to function of the gene in DCs and associated immunological reactivity, is induced in response to treatment with agents known to potentiate the ability of DCs to stimulate T cells, this property of DCs being central to their key regulatory role within the immune system. The induction of GID3 gene (polynucleotide) expression in response to stimulation of DC is shown in FIG. 3 GID3 genes (polynucleotides) and GID3 polypeptides (protein) described herein are GID3 genes (polynucleotides) and GID3 polypeptides (protein) provided by the present invention.

We have also found that from cDNA synthesized from RNA of isolated human DC, recombinant clones containing copies of the transcripts of a GID4 gene may be isolated and identified; re-amplification of the identified sequences directly from cDNA from DC, and analysis of multiple clones, reveals that the consensus sequences consist of the polynucleotide sequences SEQ ID NO:11, the polypeptide-coding sequences SEQ ID NO:12, and the corresponding encoded polypeptide sequence SEQ ID NO:12 of the GID4 transcript. The latter sequence consists of a large (832 amino acid) protein containing from 6 to 8 potential transmembrane domains dispersed along the length of the polypeptide. There are 7 potential asparagine N-glycosylation sites (positions 15, 177, 266, 368, 406, 462 and 511) specific to the consensus sequence Asn-Xaa-Ser/Thr (Marshall, R. D., Annu. Rev. Biochem. 41 [1972] 673-702). Additional motifs found in GID4 include one potential cAMP/cGMP-dependent protein kinase phosphorylation site at the threonine 224 residue, according to the rule of two consecutive, N-adjacent basic residues (Fremisco, J. R. et al., J. Biol. Chem. 255 [1980] 4240-4245; Glass, D. B. and S. B. Smith, J. Biol. Chem. 258 [1983] 14797-14803; G.ass, D. B. et al., J. Biol. Chem. 264 [1986] 2987-2993); 17 potential casein kinase II phosphorylation sites (at serine or threonine residues 76, 114, 268, 337, 397, 456, 535, 603, 746, 779, 791, 792, 800, 801, 814, 820 and 824) according to the consensus (S/T)(x2)(D/E) rule (Pinna, L. A., Biochim. Biophys. Acta 1054 [1990] 267-289); 8 potential protein kinase C phosphorylation sites (at serine or threonine residues 25, 222, 227, 253, 388, 424, 700 and 768) according to the consensus (S/T)(x)(R/K) rule (Woodget, J. R., et al., Eur. J. Biochem. 161 [1986] 177-148; Kishimoto, A., et al., J. Biol. Chem. 260 [1985] 12492-12499); 4 potential tyrosine kinase phosphorylation sites (at tyrosine residues 86, 143, 257 and 258) according to the consensus (R/K)(x2/3)(D/E)(x3/2)(Y) rule (Patschinsky, T., et al., Proc. Natl. Acad. Sci. USA 79 [1982] 973-977; Hunter, T., J. Biol. Chem. 257 [1982] 4843-4848; Cooper, J. A. et al., J. Biol. Chem. 259 [1984] 7835-7841); and 4 potential N-myristoylation sites (at glycine residues 35, 39, 621 or 787; these are internal glycine residues, and N-terminal myristoylation at any of these sites depends upon proteolytic cleavage of the GID4 protein, in this case a precursor protein, that would expose one of the glycine residues at the N-terminus that would then be subject to the action of the transfer enzyme, myristoyl CoA:protein N-myristoyl transferase) according to the consensus pattern defined by Towler, D. A. et al., Annu. Rev. Biochem. 57 [1988] 69-99 and Grand, R. J. A., Biochem. J. 258 [1989] 625-638. The GID4 protein contains a well-conserved (E=1.2e-89) large polypeptide domain from residues 348-809 termed DUF221 (a PFAM signature) that is characteristic of many hypothetical transmembrane proteins of unknown function. The sequence homology of the GID4 protein sequence to the consensus DUF221 sequence is shown in FIG. 4, 5 to 6 of the putative transmembrane domains in GID4 lie within this domain. Known examples of the DUF221-containing family of proteins include the hypothetical S. cerevisiae Ylr241w (SwissProt access number S59387), and the 107.7 KDA protein encoded by the gene ymr266w (SwissProt accession number Q03516); the hypothetical S. pombe 90.9 kda protein encoded by the gene spac2g11.09 (SwissProt access number Q09809); the 81.9 KDA protein of the plant Arabidopsis thaliana (TREMBL: Q9C8G5); the D. melanogaster 760-amino acid CG11210 protein (TREMBL:Q9V364); an hypothetical human 807-amino acid KIAA0792 protein (TREMBL:094886), and an hypothetical human 93.3/93.8 KDA protein (TREMBL:Q9P1W3 and Q9P1W1). The most closely related known human proteins are the KIAA0792 protein (TREMBL:094886; Nagase, T., et al., DNA Res. 5 [1998] 277-286), that aligns with the GID4 polypeptide sequence SEQ ID NO:12 (2.1e-174) over a 753-amino acid overlap with 59.0% identity; and the 93.3/93.8 KDA protein (TREMBL:Q9P1W3 and Q9P1W1) that aligns (4.8e-128) with 43.0% identity over an 817-amino acid overlap. DUF221-containing proteins in very distantly related organisms also display striking homology, as the D. melanogaster 760-amino acid CG11210 protein (TREMBL:Q9V364; Adams, M. D., et al., Science 287 [2000] 2185-2195), that aligns with the GID4 protein sequence SEQ ID NO:12 (8.3e-49) with 33.3% identity in 729-amino acid overlap; and the 81.9 KDA protein of the plant Arabidopsis thaliana (TREMBL:Q9C8G5; Theologis, A., et al., Nature 408 [2000] 816-820), that aligns (8.6e-14) with 23.2% identity in a 656-amino acid overlap. In addition to the overall sequence homology of these proteins, there is also a conserved structural organization, as defined by the relative position of the DUF221 domain within the protein, and the distribution of potential transmembrane domains and N-glycosylation sites; this relationship is depicted in FIG. 5. The 370 C-terminal sequences of the GID4 protein are identical to a known hypothetical protein fragment (TREMBL:Q9NSG5).

We have observed that GID4 expression in DCs, with relevance to function of the gene in DCs and associated immunological reactivity, is induced in response to treatment with agents known to potentiate the ability of DCs to stimulate T cells, this property of DCs being central to their key regulatory role within the immune system. The induction of GID4 gene expression in response to stimulation of DC is shown in FIG. 6. By homology of the GID4 protein to the S. cerevisiae Ylr241w protein, that is categorized as a member of the major facilitator superfamily of transmembrane proteins, of unknown function (Nelissen, B. et al., FEMS Microbiol. Reviews 21 [1997] 113-134), a putative activity of the GID4 protein in stimulated DCs or other cells is to participate in mediation of activation signals or other processes occurring at cellular membranes during e.g. change of cellular state as in maturation or differentiation or cellular activation, or at the interface between other cellular compartments and membrane surfaces, through concerted interactions with other cellular proteins, that are critical for said change of cellular state. In the case of DC, this change of state (e.g. cellular activation) is amplified phenotopically during interaction of DC with T cells for normal immunological responses.

In one aspect the present invention provides an isolated
BASP1 gene expressed in dendritic cells (DCs) of the immune system or
GID2 gene (polynucleotide) expressed in dendritic cells of the immune system or
GID3 gene, e.g. expressed in dendritic cells of the immune system or
GID4 gene, e.g. expressed in dendritic cells of the immune system.

In another aspect the present invention provides an isolated
BASP1 gene comprising the sequence of SEQ ID NO:1 or
GID2 gene comprising the sequences SEQ ID NO:2, and/or SEQ ID NO:3 and/or SEQ ID NO:5 or
GID3 gene comprising the sequence SEQ ID NO:7, e.g. containing the coding sequence SEQ ID NO:8 or
GID4 gene comprising the sequence SEQ ID NO:11, e.g. containing the coding sequence SEQ ID NO:12.
A BASP1 gene expressed in DCs of the immune system according to the present invention encodes a BASP1 polypeptide, e.g. of the SwissProt P80723 entry. A BASP1 polypeptide of the present invention includes a polypeptide of the SwissProt P80723 entry.
A GID2 polynucleotide (gene) provided by the present invention encodes GID2 polypeptides (protein), e.g. of sequences GID2.1 SEQ ID NO:4 and/or GID2.2 SEQ ID NO:6.
A GID2 polypeptide of the present invention thus includes a polypeptide of the sequences GID2.1 SEQ ID NO:4 and/or GID2.2 SEQ ID NO:6.
A GID3 gene provided by the present invention encodes a GID3 polypeptide, e.g. of SEQ ID NO:9. A GID3 polypeptide of the present invention includes a polypeptide of SEQ ID NO:9.
A GID4 gene (polynucleotide) according to the present invention encodes a GID4 polypeptide, e.g. of the sequence SEQ ID NO:12. A GID4 polypeptide (protein) of the present invention includes a polypeptide of the sequences SEQ ID NO:12.

In another aspect the present invention provides an isolated polypeptide e.g. expressed in dendritic cells of the immune system of
BASP1, e.g. of the SwissProt P80723 entry encoded by a BASP1 gene or
GID2, e.g. of SEQ ID NO:4 and/or SEQ ID NO:6 e.g. encoded by a GID2 gene or
GID3 polypeptide, e.g. of SEQ ID NO:9, e.g. encoded by a GID3 gene, e.g. or
GID4 polypeptide, e.g. of SEQ ID NO:12, e.g. encoded by a GID4 gene.

“Polynucleotide”, if not otherwise specified herein, includes any polyribonucleotide or polydeoxyribonucleotide, which may be unmodified RNA or DNA, or modified RNA or DNA, including, and without limitation, single- and double-stranded RNA or DNA, and RNA or DNA that is a mixture of single- and double-stranded regions.

“Polypeptide”, if not otherwise specified herein, includes any peptide or protein comprising two or more amino acids joined to each other by peptide bonds.

The genes (polynucleotide) expressed in DCs according to the present invention includes the polynucleotide of corresponding sequences as set out in TABLE 1 (e.g. SEQ ID NO:1, SEQ ID NO:2, SEQ ID NO:3, SEQ ID NO:5, SEQ ID NO:7, SEQ ID NO:8, SEQ ID NO:10 and SEQ ID NO: 11) and variants thereof, e.g. that may occur naturally in DC; e.g. including allelic variants, and transcriptional and post-transcriptionally modified variants thereof, and/or their complements; encoding a polypeptide sequence of the genes as set out in TABLE 1 (e.g. SEQ ID NO: 4, SEQ ID NO: 6, SEQ ID NO: 9 and SEQ ID NO:12) and including allelic variants thereof, and variations due to transcriptional and post-transcriptional differences in the polynucleotides, and variations due to post-translational modification. The polynucleotides and polypeptides according to the present invention in DCs of the immune system include sequences expressed in cells closely related to DC and of a similar immunological function according to the present invention; and e.g. orthologous variants expressed in DC of the immune system in laboratory animals commonly used in pharmaceutical research, and cells closely related to DCs in those species according to the present invention, where said variations are due to mechanisms commonly known to occur during species divergence; and e.g. allelic, transcriptional and post-transcriptional, and post-translational variants of said orthologous sequences.

“Allelic variant” of the gene of the present invention is a form of the polynucleotide and polypeptide of the gene existent within a given species that differs from the SEQ ID NO:1, SEQ ID NO:2, SEQ ID NO:3, SEQ ID NO:5, SEQ ID NO:7, SEQ ID NO:8, SEQ ID NO:10 and SEQ ID NO: 11 and the SwissProt P80723 entry, respectively, and the complementary polynucleotide sequences, where the differences may be attributed to mutation that has occurred during species evolution by mechanisms that are commonly known. Typically, allelic variants have identity of greater than 95%, e.g. such as 97% or 99%, and less than 100%, over the entire sequence length; said identity being calculated as the percentage of identical nucleotide or amino acid residues at corresponding positions within an optimal linear alignment of the entire lengths of the sequences.

“Transcriptional variant” of the gene is a polynucleotide and an encoded polypeptide existent within a given species that differs from the sequences as given in TABLE 1 and e.g. the SwissProt P80723 entry, respectively, and the corresponding polynucleotide complementary sequences, by containing additional sequence segments, or by lacking sequence segments, at the beginning or end of an optimal linear alignment of the entire lengths of the sequences; where said variation may be attributed to the position of initiation and/or termination of RNA synthesis from the gene chromosomal template by mechanisms that are commonly known.

“Post-transcriptional variant” of the gene is a polynucleotide and an encoded polypeptide of the gene existent within a given species that differs from the given sequences e.g. SEQ ID NO:1, SEQ ID NO:2, SEQ ID NO:3, SEQ ID NO:5, SEQ ID NO:7, SEQ ID NO:8, SEQ ID NO:10 and SEQ ID NO: 11 and the SwissProt P80723 entry, respectively, and the complementary polynucleotide sequences, by containing additional sequence segments, or by lacking sequence segments, at the beginning or end or within an optimal linear alignment of the entire lengths of the sequences; where said variant may be attributed to the excision and splicing of RNA sequence segments synthesized from the chromosomal template by mechanisms that are commonly known.

“Post-translational variant” of the gene is a polypeptide sequence of the gene existent within a given species that differs from the given sequences and e.g. SwissProt P80723 entry by lacking sequence segments, or containing additional sequence segments, at the beginning or end of an optimal linear alignment of the entire lengths of the sequences, where the difference in sequence length may be attributed to specific proteolytic modification; or that differs from the given sequences and e.g. SwissProt P80723 entry by containing biochemical alterations of specific amino acyl residues contained within that sequence, that may include e.g. phosphorylation, myristoylation, acetylation, e.g. an alteration among the many types of biochemical alterations of proteins that are commonly known to exist.

“Orthologous variant” of the gene is a polynucleotide and an encoded polypeptide of the gene that exists in a second species, where the differences from the given sequences e.g. SEQ ID NO:1, SEQ ID NO:2, SEQ ID NO:3, SEQ ID NO:5, SEQ ID NO:7, SEQ ID NO:8, SEQ ID NO:10 and SEQ ID NO: 11 and the SwissProt P80723 entry, respectively, and the complementary polynucleotide sequences, seen in an optimal linear alignment of the entire lengths of the sequences may be attributed to mutation that has occurred during species divergence during evolution by mechanisms that are commonly known.

“Alignment” is the placing together of two linear sequences according to the identity or relatedness, or complementarity, of the constituent single nucleotidyl or amino acyl residues along those linear sequences, or segments or blocks of those residues, with allowance for minimal single-residue or segment deviations, such that the overall relatedness of the sequences may be determined. Said relatedness is a measure of the identity of polynucleotide or polypeptide sequences and may be calculated by conventional methods, employing e.g. commercially available computer programs.

A polynucleotide according to the present invention, e.g. including (existing) variants or parts thereof, may be obtained from natural sources as a molecular clone from cDNA or a cDNA library derived from mRNA from DCs or other cells, or from genomic DNA, by using standard cloning, screening and sequencing methods, e.g. identification of a BASP1 polynucleotide sequence from a cDNA library by the expressed sequence tag (EST) analysis (Adams, M. D. et al., Science 252 [1991] 1651-1656; Adams, M. D. et al., Nature 355 [1992] 632-634; Adams, M. D. et al., Nature 377 [1995] Suppl.:3-174). A BASP1 polynucleotide, or variants or parts thereof, may also be synthesized according to a method as conventional. A BASP1 polypeptide according to the present invention, e.g. including (existing) variants or parts thereof, may be obtained from natural sources, e.g. may be isolated from cells or tissues; a polypeptide, or variants or parts thereof, may also be synthesized according to a method as conventional.

A polynucleotide (gene) according to the present invention may be incorporated as part of a recombinant molecule as appropriate to the use of recombinant molecules according to skills in the art, e.g. to confer properties of utility or of altered gene function according to the present invention, by combining the gene sequences with exogenous DNA sequences (i.e., DNA sequences that are not contained within polynucleotides according to the present invention) that confer such properties, e.g. according to methods as conventional. If a polynucleotide is used for the production of a polypeptide, the polynucleotide sequence may include other sequences, e.g. the coding sequence for the mature polypeptide (or fragment thereof) by itself; or the polypeptide coding sequences (or fragment thereof) fused in continuous reading frame with exogenous coding sequences, such as those encoding a leader or secretory sequence, a pre-, or pro- or prepro-protein sequence, or other fusion peptide portions, e.g. such that are commonly used for such purposes. For example, an exogenous marker sequence that facilitates purification of the recombinant polypeptide can be combined. The marker sequence may be as appropriate, e.g. including an hexa-histidine peptide, as provided in the pQE vector (Qiagen, Inc.) and e.g. described in Gentz et al., Proc Natl Acad Sci USA 86 [1989] 821-824), or e.g. an hemagglutinin (HA) tag. A recombinant polynucleotide according to the present invention may also contain non-coding nucleotide sequences, e.g. non-translated sequences, such as splicing and polyadenylation signals, ribosome binding sites, localization sequences, and sequences that stabilize or destabilize mRNA, e.g. any of the several non-coding sequences commonly known to confer regulatory properties as appropriate, e.g. according to skills of the art; non-coding nucleotide sequences may occur naturally in transcripts or transcriptional variants of the gene according to the present invention, or polynucleotide sequences as appropriate, e.g. according to methods as conventional.

A recombinant molecule containing a gene sequences used for the production of an appropriate polypeptide or polyribonucleotide or the complementary polyribonucleotide, or parts thereof, may also include specific mutations, e.g. as occur naturally in allelic or homologous or orthologous variants of the gene, e.g. as may be introduced in the gene sequence artificially by methods as conventional, that result in modified functional properties of the corresponding polypeptide or polyribonucleotide or the complementary polyribonucleotide according to the present invention.

In another aspect the present invention provides a vector containing a gene (polynucleotide) according to the present invention; e.g. and/or a polypeptide according to the present invention.

A vector containing a sequence as set out in TABLE 1 may be produced as appropriate, e.g. according to a method as conventional. A vector containing a sequence may be produced e.g. to artificially add specific properties to the sequence, such as properties changing physical transfer, autonomous or cell-dependent replication, expression and selection. Such vector may e.g. be produced by combining the polynucleotide sequence with exogenous DNA sequences that confer desired properties, e.g. according to a method as conventional, e.g. using an appropriate vector that may be obtained commercially. Expression properties of the polynucleotide contained within the vector may be e.g. determined by incorporating the naturally occurring gene promoter, or promoters of other genes, e.g. genes of eukaryotes, of prokaryotes or of viruses, as is appropriate for the desired expression; e.g. according to skills in the art, e.g. according to a method as conventional.

A vector containing a gene according to the present invention may be useful, e.g. to obtain an expression system which is able to produce a polynucleotide or a recombinant polypeptide, polyribonucleotide or its complement, e.g. in a host cell, such as in a compatible host cell. For example, a BASP1, GID2, GID3 or GID4 or recombinant BASP1, GID2, GID3 or GID4 polypeptide may be synthesized according to the present invention in a genetically engineered host cell; e.g. by use of a vector comprising a BASP1, GID2, GID3 or GID4 gene to incorporate into the host cell an expression system for the corresponding gene, e.g. for the production of a BASP1 polypeptide, such as a BASP1 polypeptide of the SwissProt P80723 entry, or e.g. a part or variant(s) thereof according to the present invention. Cell-free transcription or translation systems may also be used to produce a polynucleotide or polypeptide, e.g. using RNAs derived from a recombinant DNA vector containing the polynucleotide sequences according to the present invention; e.g. according to a method as conventional.

A vector containing a gene promoter sequence, i.e. sequences obtained from chromosomal DNA lying proximal to the chromosomal template of a polynucleotide that control transcriptional expression of a gene according to the present invention, may be produced as appropriate. Such a vector may also contain polynucleotides according to the present invention, e.g. and as described above, i.e. the vector physically links sequences specifying the properties of a gene transcriptional expression with those specifying the gene activity; or may also contain exogenous sequences comprising an indicator molecule that may be easily and quantifiably detected, i.e. the vector physically links sequences specifying the properties of the gene transcriptional expression with those specifying a simple indicator of the gene promoter activity.

In another aspect the present invention provides an expression system comprising a gene (polynucleotide) according to the present invention, e.g. an isolated polynucleotide that has been modified as described above; or an isolated gene promoter sequence, e.g. as described above; contained as part of a recombinant vector, e.g. comprising a polynucleotide according to the present invention as a part of an expression vector, wherein said expression system or part thereof is capable of producing a corresponding polynucleotide and/or polypeptide according to the present invention, e.g. or said expression system or part thereof is subject to transcriptional control by an appropriate promoter sequences, e.g. as described above, e.g. comprising an expression system or part thereof capable of expression any of the recombinant vectors described above; when said expression system or part thereof is present in a compatible host cell.

In another aspect the present invention provides a process for the production of an altered host cell comprising a polynucleotide according to the present invention, e.g. as defined above,
or the complementary polynucleotide sequence or variants thereof,
or modifications thereof,
or parts of such polynucleotides,
or recombinant molecules containing such polynucleotides,
   e.g. as described above; or
of a host cell that contains such polynucleotides that are capable of producing a polypeptide corresponding to the specific polynucleotide; or
of a host cell that contains a polypeptide of the above mentioned or variants thereof, or modifications or parts thereof;
e.g. an altered host cell that contains and is capable of expressing any of the expression systems described above;
which process comprises transferring said polynucleotides or polypeptides into the host cells; e.g. where the process may be used to transfer or modify the activity of the gene according to the present invention in said host cells.

Transfer includes e.g. transformation, transfection, tranvection, electroporation or microinjection, e.g. according, e.g. analogously, to methods as conventional.

In another aspect the present invention provides a process for the production of a stable transgenic host cell that contains a gene (polynucleotide) according to the present invention as part of an expression system, or any of the expression systems described above, comprising transferring DNA of a suitable recombinant vector containing a polynucleotide into a host cell, e.g. introducing a gene expression system into a suitable host cell, and isolating the cells that maintain the transferred DNA stably, e.g. said cells being defined as stable transgenic host cells.

The stable transgenic host cell according to the present invention under appropriate culture conditions produces a corresponding polyribonucleotide, or a complement therof, e.g. of given sequences, or variants or modifications or parts thereof, e.g. as described above. The stable transgenic host cell according to the present invention may be capable of producing a polypeptide that corresponds to said polyribonucleotide. That process according to the present invention may be used to stably transfer or modify the activity of the gene according to the present invention under appropriate conditions in said transgenic host cells.

In another aspect the present invention provides an isolated host cell comprising an expression system according to the present invention, e.g. a stable gene-containing transgenic host cell; e.g. produced and isolated by a process described above.

In another aspect the present invention provide a process for producing a polynucleotide and/or a polypeptide according to the present invention comprising culturing an isolated, altered or stable transgenic host cell as described above, that supports an expression system according to the invention, under conditions sufficient for the production of a polynucleotide and/or polypeptide of the present invention, and recovering said polynucleotide or polypeptide from the culture; e.g. wherein the sequence of the recovered polynucleotide or polypeptide, and the composition of the recovered material, is determined by the nature of the polynucleotide within a vector comprising the expression system, the action of the supporting host cell, and the biochemical methods of isolation of the material.

Introduction of polynucleotides into host cells may be e.g. effected as appropriate, e.g. according to a method as conventional, e.g. according to Davis et al., Basic Methods in Molecular Biology (1986); Sambrook et al., Molecular Cloning: A Laboratory Manual, 2nd Ed., Cold Spring Harbor Laboratory Press, Cold Spring Harbor, N.Y. (1989), e.g. including methods as calcium phosphate-mediated transfection, DEAE-dextran-mediated transfection, transvection, microinjection, cationic lipid-mediated transfection, electroporation, transduction, scrape loading, ballistic introduction, infection.

Examples of appropriate host cells include e.g. bacterial cells, such as streptococci, staphylococci, E. coli, Streptomyces and Bacillus subtilis cells; fungal cells, such as yeast and Aspergillus cells; insect cells such as Drosophila S2 and Spodoptera Sf9 cells; isolated animal cells such as CHO, COS, HeLa, C127, CCL39, 3T3, BHK, HEK293 and Raji lymphoma cells; and plant cells.

Appropriate expression systems include e.g. chromosomal, episomal and virus-derived systems, e.g., vectors derived from bacterial plasmids, from bacteriophage, from transposons, from yeast episomes, from insertion elements, from yeast chromosomal elements, from viruses such as baculoviruses, papova viruses such as SV40, vaccinia viruses, adenoviruses, fowl pox viruses, pseudorabies viruses and retroviruses, and vectors derived from combinations thereof, such as those derived from plasmid and bacteriophage genetic elements, such as cosmids and phagemids. An expression systems may contain control regions that regulate as well as engender expression. Generally, any system or vector suitable to maintain, propagate or express polynucleotides and polypeptides in a host may be used. The appropriate nucleotide sequence may be inserted into an expression system according to a method as conventional, e.g. according to Sambrook et al., Molecular Cloning: A Laboratory Manual (supra).

A polynucleotide or polypeptide according to the present invention, expressed by a host cell that has been engineered to support an expression system according to the present invention, may be isolated from the host cells in sufficient yield and purity by any appropriate method, e.g. including methods of (biochemical) extraction and separation, e.g. such as according to conventional methods. The yield and purity may be that suitable for the purposes for which the isolated material may be used. For example, a polynucleotide of the present invention may be isolated as a double-or single-stranded DNA recombinant phagemid molecule from bacterial host cells, or as a polyribonucleotide, dependent upon the nature of the vector comprising the expression system, e.g. for the purpose of obtaining larger amounts of purified polynucleotide. Polynucleotide that is incorporated in a suitable system expressing infectious or noninfectious viral particles may be isolated in an appropriate form, e.g. in a form appropriate for biological experiments or screening assays designed to detect modulation of gene activity according to the present invention. A corresponding polypeptide may be isolated in an appropriate form, e.g. in a form appropriate for biological experiments or screening assays designed to detect modulation of gene activity according to the present invention, or, in some cases, the host cells expressing the corresponding polypeptide may be used directly for such assays. The polypeptide may be used to generate antibodies in commonly used laboratory animals.

Isolation and purification of a polypeptide may be performed as appropriate, e.g. according to methods as conventional; e.g. including combinations of appropriate methods. Appropriate methods e.g. include direct harvesting from expression host cell cultures, extraction, such as detergent extraction acid extraction; centrifugation, such as ultracentrifugation, density gradient centrifugation; precipitation, such as ammonium sulfate or ethanol precipitation; chromatography, such as including anion or cation exchange chromatography, phosphocellulose chromatography, hydrophobic interaction chromatography, affinity chromatography, hydroxylapatite chromatography, lectin chromatography, high performance liquid chromatography. If a polypeptide according to the invention is denatured during isolation and or purification, regeneration of the active conformation, e.g. refolding of a denaturated form of the polypeptide to a non-denatured form, may be carried out as appropriate, e.g. according to a method as conventional. “Isolated and purified”, if not otherwise specified herein, includes the meaning “separated from the coexisting material”, e.g. “altered by the hand of man” from the natural state.

A polynucleotide or polypeptide or vector or expression system or host cell according to the present invention may be used as a research reagent, and as material for the discovery of treatments for the animal and human diseases. Such diseases include diseases in which immunological reactions are a primary component of etiology, progression and/or exacerbating symptoms associated with the disease; and include e.g. acute and chronic inflammatory diseases, including for example inflammatory skin diseases such as allergic and atopic dermatitis, and chronic inflammatory diseases whose symptoms appear in other tissues such as intestine and colon, lung and vascular tissue; e.g. abnormal immune reactivity in autoimmune diseases, or undesired immunological reactions associated with therapeutic treatment of diseases as in transplant rejection crises; e.g. immune hypo-reactivity or suppression in diseases such as cancer, and that associated with persistent viral or microbial infection. E.g. an antagonist of the interaction between the polypeptide and calmodulin may disrupt calcium-mediated transduction pathways necessary for dendritic cell activation in immunological reactions associated with diseases, e.g. diseases as described above. E.g. alternatively, antagonists of the induction steps leading to overexpression of the gene according to the present invention in dendritic cells during immune activation may block calcium-mediated transduction pathways in these cells. Activated dendritic cells in which the gene activity is antagonized may modulate or direct T cell responses associated with amplification of immune stimulation signals.

In another aspect the present invention provides the use of a gene according to the present invention as a diagnostic reagent.

Detection of abnormal expression, or an allelic form, of the gene of the present invention associated with a dysfunction will provide a diagnostic tool, e.g. in a diagnostic assay, that may add to or define a diagnosis of a disease or susceptibility to a disease. Allelic variants of the gene may be detected in DNA of individuals, and expression of the gene may be detected in cDNA, or as protein using antibodies or other methods, e.g. according to methods as conventional.

DNA or RNA or protein for diagnosis may be obtained from a subjects cells, such as from blood, urine, saliva, tissue biopsy or autopsy material. Genomic DNA may be used directly for detection or may be amplified enzymatically by using PCR or other amplification techniques prior to analysis. RNA or cDNA may also be used in the analysis similarly. Deletions and insertions may be detected by a change in size of the amplified product in comparison to that from a normal subject. Point mutations may be identified by hybridizing amplified DNA to labeled BASP1 gene nucleotide sequences. Perfectly matched sequences may be distinguished from mismatched duplexes, e.g. by RNase digestion or by differences in melting temperatures. DNA sequence differences may also be detected by alterations in electrophoretic mobility of DNA fragments in gels, with or without denaturing agents, or by direct DNA sequencing, e.g. according to Myers et al., Science (1985) 230:1242. Sequence changes at specific locations may also be revealed by nuclease protection assays, such as RNase and S1 protection or the chemical cleavage method, e.g. according to Cotton et al., Proc Natl Acad Sci USA (1985) 85: 4397-4401. An array of oligonucleotides probes comprising the polynucleotide sequence of the present invention or fragments thereof may be constructed to conduct efficient screening of e.g. specific allelic polymorphisms. Array technology methods may e.g. be used to address a variety of questions in molecular genetics including gene expression, genetic linkage, and genetic variability, e.g. according to M. Chee et al., Science (1996) 274: 610-613.

Decreased or increased expression may be determined from RNA expressed in a subject's cells, e.g. according to a method as conventional for the quantitation of polynucleotides, such as PCR, RT-PCR, RNase protection, Northern blotting and other hybridization methods. Assay techniques that may be used to determine levels of a protein, such as a polypeptide, in a subject's cells may be carried out, e.g. according to a method as conventional. Such assay techniques include radioimmunoassays, competitive binding assays, Western Blot analysis and ELISA assays using antibodies prepared against the polypeptide; e.g. identification in two-dimensional separation gels, and liquid phase separation techniques followed by sequence or molecular weight analysis.

A diagnostic assays offer a process for diagnosing or determining a susceptibility to diseases, e.g. acute and chronic inflammatory diseases, including for example inflammatory skin diseases such as allergic and atopic dermatitis, and chronic inflammatory diseases whose symptoms appear in other tissues such as intestine and colon, lung and vascular tissue; e.g. abnormal immune reactivity in autoimmune diseases, or undesired immunological reactions associated with therapeutic treatment of diseases as in transplant rejection crises; e.g. immune hypo-reactivity or suppression in diseases such as cancer, and that associated with persistent viral or microbial infection; through detection of allelic polymorphisms and abnormal expression of the gene of the present invention, e.g. according to a method as described above, e.g. including a method as conventional.

Thus in another aspect, the present invention provides a diagnostic kit for a disease or susceptibility to a disease as described above, comprising:
(a) a gene according to the present invention, e.g. including a variant or fragment as described;
(b) a nucleotide sequence complementary to that of (a);
(c) a polypeptide according to the present invention, e.g. including a polypeptide of an amino acid sequence which has at least 80% identity thereto, e.g. including a fragment or a variant of a polypeptide of an amino acid sequence which has at least 80% identity to said polypeptide of the present invention; or
d) an antibody to a polypeptide according to the present invention.

In any such kit, (a), (b), (c) or (d) may comprise a substantial component, including an appropriate environment of a sample to be tested, e.g. and appropriate means to determine the effect of any of a), b), c) or d) in a sample to be tested.

A gene (polynucleotide) according to the present invention may also be useful for chromosome identification. The sequence is specifically targeted to and can hybridize with a particular location on an individual human chromosome, and/or the sequences may be identified in GenBank chromosomal sequence database entries. Susceptibilities to many diseases, including e.g. viral diseases and arthritis, have been mapped according to such methods in rodents. The mapping of relevant sequences to chromosomes according to the present invention is an important first step in correlating those sequences with gene associated disease. Once a sequence has been mapped to a precise chromosomal location, the physical position of the sequence on the chromosome may be correlated with genetic map data. Such data are found e.g. in V. McKusick, Mendelian Inheritance in Man (available on-line through Johns Hopkins University Welch Medical Library). The relationship between genes and diseases that have been mapped to the same chromosomal region may be identified through linkage analysis (coinheritance of gene sequences that are physically located within a defined chromosomal region). The differences in the cDNA or genomic sequence between affected and unaffected individuals may also be determined. If a mutation is observed in some or all of the affected individuals but not in any normal individuals, then the mutation is likely to be a causative factor of the disease, or susceptibility to the disease, e.g. a factor of a mechanism that is involved in relevant biological activities necessary for the development or progression of an indication of the disease.

In another aspect the present invention provides genetically modified animals, e.g. useful in laboratory research for gene function studies according to the present invention in vivo, e.g. mice or rats, e.g. lower eukaryotic organisms such as D. melanogaster or Caenorhabditis, wherein orthologous or homologous gene sequences at the orthologous gene locus or at other loci are modified; e.g. created by homologous or non homologous recombination methods in germ line cells or other appropriate cells, and commonly used breeding techniques. For reviews of such techniques, see e.g. Capecchi, M., Science 244 [1989] 1288-1292; Benoist, C. and D. Mathis, Curr. Opin. Immunol. 5 [1993] 900-902; Charreau, B. et al., Transgenic Res. 5 [1996] 223-234; Moreadith, R. W. and N. B. Radford, J. Mol. Med. 75 [1997] 208-216; Nomura, T., Lab. Anim. Sci. 47 [1997] 113-117; Cohen-Tannoudji, M. and C. Babinet, Mol. Hum. Reprod. 4 [1998] 929-938. Such genetic modifications may include e.g. substitutions of orthologous or homologous gene sequences, e.g. or fragments or variants thereof, e.g. such as described herein, that may be part of an expression system, e.g. as described above, at the orthologous gene locus or at other loci, e.g. insertions of any of the said sequences at the orthologous gene locus or at other loci, e.g. deletions of the orthologous gene locus. Such genetic modifications may include stable alterations as described, or may include conditional modifications wherein e.g. the specific deletion event or e.g. the expression of the said inserted or substituted sequences may occur in only selected cells, or in selected cells upon an appropriate treatment of the genetically modified organisms. Any of the genetic variants described above may be produced in laboratory animals that have been inbred or engineered at other loci to effect specific immunological responses, e.g. within a genetic background that is highly susceptible to a specific challenge, e.g. to effect a more reproducible or penetrating immunological response following specific challenge, e.g. to effect immunological reactions in response to challenge that may be more characteristic of those observed in humans, or that have been inbred or engineered at other loci to effect specific responses in other biological systems in order to study complex organismal interactions in vivo.

In another aspect the present invention provides genetically modified animals, e.g. useful in laboratory research for gene function studies in vivo, e.g. genetically modified mice or rats, e.g. genetically modified lower eukaryotic organisms such as D. melanogaster or Caenorhabditis, wherein orthologous or homologous gene sequences at the orthologous gene locus or at other loci are modified (in respect with normal unmodified gene sequences); e.g. which genetically modified animals may be created by homologous or nonhomologous recombination methods in germ line cells or other appropriate cells, and commonly used breeding techniques.

For reviews of techniques for genetic modification of laboratory animals see e.g. Capecchi, M., Science 244 [1989] 1288-1292; Benoist, C. and D. Mathis, Cur. Opin. Immunol. 5 [1993] 900-902; Charreau, B. et al., Transgenic Res. 5 [1996] 223-234; Moreadith, R. W. and N. B. Radford, J. Mol. Med. 75 [1997] 208-216; Nomura, T., Lab. Anim. Sci. 47 [1997] 113-117; Cohen-Tannoudji, M. and C. Babinet, Mol. Hum. Reprod. 4 [1998] 929-938. Such genetic modifications include e.g. substitutions of orthologous or homologous gene sequences, or fragments or variants thereof, that may be part of an expression system according to the present invention, at the orthologous gene locus or at other loci by modified sequences, e.g. insertions of a modified sequence at the orthologous gene locus or at other loci, e.g. deletions of the orthologous gene locus. Such genetic modifications may include stable alterations, or may include conditional modifications wherein e.g. the specific deletion event or e.g. the expression of the said inserted or substituted sequences may occur in only selected cells, or in selected cells upon an appropriate treatment of the genetically modified organisms. Any of genetic variants, e.g. such as described above, may be produced in laboratory animals that have been inbred or engineered at other loci to effect specific immunological responses, e.g. within a genetic background that is highly susceptible to a specific challenge, e.g. to effect a more reproducible or penetrating immunological response following specific challenge, e.g. to effect immunological reactions in response to challenge that may be more characteristic of those observed in humans, or that have been inbred or engineered at other loci to effect specific responses in other biological systems in order to study complex organismal interactions in vivo.

In another aspect the present invention provides an (isolated) antibody against a polypeptide of the present invention, e.g. of polypeptide sequences as listed in TABLE 1, e.g. including fragments or variants thereof, e.g. as described herein.

A polypeptide of the present invention, or cells expressing such a polypetide, may be used as an immunogen to produce antibodies immunospecific for a corresponding polypeptide. The term “immunospecific” means that the antibodies have substantially greater affinity for the polypeptides than their affinity for other related polypeptides in the prior art.

Antibodies generated against a polypeptide of the present invention may be obtained e.g. by administering the polypeptide or an epitope-bearing fragment, analog or expression cell to an animal, preferably non-human, using routine protocols. For preparation of monoclonal antibodies, any technique which provides antibodies, e.g. produced by continuous cell line cultures may be used, e.g. including the hybridoma technique (Kohler, G. and C. Milstein, Nature 256 [1975] 495-497), the trioma technique, the human B-cell hybridoma technique (Kozbor et al., Immunology Today 4 [1983] 72) and the EBV-hybridoma technique (Cole et al., In Monoclonal Antibodies and Cancer Therapy, pp. 77-96, Alan R. Liss, Inc. [1985]). Techniques for the production of single chain antibodies (U.S. Pat. No. 4,946,778) can also be adapted to produce single chain antibodies to GID2 polypeptides. Also, transgenic mice, or other organisms including other mammals, may be used to express humanized antibodies.

Antibodies as described above may be used as appropriate, e.g. in the isolation or in the identification of a host cell expressing a polypeptide of the present invention; e.g. for the purification of a polypeptide of the present invention by affinity chromatography. Antibodies against a polypeptide of the present invention may also be useful in the treatment of diseases, e.g. include diseases in which immunological reactions are a primary component of etiology, progression and/or exacerbating symptoms associated with the disease.

An antibody according to the present invention includes e.g. a bispecific antibody binding to the polypeptide of the present invention and to a polypeptide of a viral, bacterial or eukaryotic parasite, or the polypeptide encoded by a second host gene that may be expressed either in the same cell as the polypeptide of the present invention or in a second cell.

In another aspect the present invention provides a bispecific antibody binding to the polypeptide of the present invention and binding to a second polypeptide of a viral, bacterial or eukaryotic parasite, e.g. wherein said second polypeptide is encoded by a second host gene that may be expressed either in the same cell as the polypeptide of the present invention or in a second cell, including e.g. a cancer cell.

Instead of a bispecific antibody also another bispecific reagent having the ability to bind to the polypeptide of the present invention and to a polypeptide of a viral, bacterial or eukaryotic parasite, or to the polypeptide encoded by a second host gene that may be expressed either in the same cell as the polypeptide of the present invention or in a second cell, including e.g. a cancer cell, may be used.

In another aspect the present invention provides a bispecific reagent which is other than an antibody, having the ability to bind to a polypeptide of the present invention and a viral, bacterial or eukaryotic parasite or to a cancer cell.

In another aspect the present invention provides a method for inducing an immunological response in a mammal that comprises inoculating said mammal with a polypeptide of the present invention in an amount sufficient to produce antibody and/or T cell immune response(s) to protect said mammal from diseases, e.g. including diseases in which immunological reactions are a primary component of etiology, progression and/or exacerbating symptoms associated with the disease.

In another aspect the present invention provides a method of inducing an immunological response in a mammal that comprises delivering a polypeptide of the present invention via a vector directing expression of a corresponding polynucleotide in vivo in order to induce an immunological response to produce antibody and/or T cell immune response(s) to protect said mammal from diseases; and, in another aspect, an immunological/vaccine formulation (composition) that, when introduced into a mammal, induces an immunological response in that mammal to a polypeptide of the present invention, wherein said formulation comprises a polypeptide of the present invention, or an expression vector comprising a polynucleotide of the present invention, or host cells comprising cells from the mammal that is treated that contain an expression vector comprising a polynucleotide of the present invention.

A vaccine formulation may further comprise a suitable carrier. Since a polypeptide according to the present invention may be broken down in the digestive system, said vaccine formulation is preferably administered parenterally (including subcutaneously, intramuscularly, intravenously, intradermally, etc. by injection). Immunological/vaccine formulations suitable for parenteral administration include aqueous and non-aqueous sterile injection solutions that may contain anti-oxidants, buffers, bacteriostatic agents and solutes that render the formulation isotonic with the blood of the recipient; and aqueous and non-aqueous sterile suspensions that may include suspending agents or thickening agents. The formulations may be presented in unit-dose or multi-dose containers, for example sealed ampoules and vials, and may be stored in a freeze-dried condition requiring only the addition of the sterile liquid carrier immediately prior to use. The vaccine formulation may further comprise additives, e.g. additives according, e.g. analogously, to conventional additives in vaccine formulations, and may be in the form of appropriate systems, e.g. including oil-in-water systems. The dosage will depend e.g. on the specific activity and stability, and may be readily determined by routine experimentation.

In another aspect the present invention provides a screening assay for agonists and antagonists of polypeptide activity and expression in dendritic cells of the immune system. A polypeptide of the present invention may be responsible for many biological functions, including many pathologies. Accordingly, it is desirable to find compounds and drugs which stimulate the activity of a polypeptide of the present invention or expression of the gene of the present invention on the one hand (agonists), and which can inhibit the function of a polypeptide of the present invention or expression of the gene of the present invention on the other hand (antagonists). A polypeptide or functional mimetics, e.g. according to Coligan et al., Current Protocols in Immunology 1(2):Chapter 5(1991), may be used to assess the binding of agonists or antagonists of the polypeptide, e.g. in cells, cell-free preparations, chemical libraries, and natural product mixtures, e.g. in a screening assay.

Screening procedures may involve the production of appropriate cells which express the polypeptide of the invention. Appropriate cells include cells e.g. from mammals, yeast, or Drosophila. Cells expressing the polypeptide of the present invention may be contacted with (exposed to) a test compound to observe stimulation or inhibition of a functional response; or effects on levels of polypeptide expression or biochemical activity, or of a marker gene whose expression is subject to control by transcriptional elements controlling gene expression according to the invention. Alternatively purified polypeptide, or mixtures containing other polypeptides that interact physically, may be contact (exposed) in solution with (to) a test compound to observe binding to the polypeptide, or effects of the compound on polypeptide binding to other polypeptides in the mixture.

A screening assay may be used in cells expressing a polypeptide of the present invention to test whether the candidate compound results in an alteration of signal generated by the action of the polypeptide, e.g. using detection systems appropriate to the cells expressing the polypeptide. Such an effect is referred to as “downstream” of the action of the polypeptide. For example, in a cell supporting an expression system comprised of the polypeptide of the present invention, preferably in a dendritic cell or a cell of similar properties according to the invention, an inhibitor of the activity of the polypeptide would also effect inhibition of the activity of a second cellular gene product that is dependent upon the polypeptide activity; most preferably assay of the activity of the second gene product is suitable to specific high throughput assay, e.g. may be readily determined by sensitive e.g. calorimetric, fluorescence, phosphorescence, radiometric or antibody techniques, i.e. according to skills in the art, e.g. by a method as conventional. A second type of screening assay referred to as “reporter gene assay” may be used in cells supporting an expression system according to the invention, that comprises a vector wherein transgene expression is controlled by the natural gene promoter, and the transgene comprises a recombinant polypeptide of the present invention fused to a marker gene that may be detected according to its level of expression, according to the invention. For example, in a cell supporting an expression system comprised of the promoter and a recombinant polypeptide transgene fused to the E. coli β-galactosidase polypeptide, preferably in a dendritic cell or a cell of similar properties according to the present invention, an inhibitor of the expression of the gene according to the present invention will be mimicked in the assay by an affect upon the transgene expression, that may be readily measured e.g. by the β-galactoside esterase enzymatic activity of the β-galactosidase fusion polypeptide; many such fusion marker genes are known, as e.g. other enzymes such as luciferase and alkaline phosphatase, as e.g. an epitope tag that may be detected with an appropriate antibody, i.e. according to skills in the art using conventional methods.

Agonists and antagonists of a polypeptide of the present invention, e.g. including (poly)peptides, monoclonal antibodies, low molecular weight chemical compounds, antisense oligonucleotides, may have immune modulatory activities and may be used in the treatment of diseases, e.g. such as in acute and chronic inflammatory diseases, including for example inflammatory skin diseases such as allergic and atopic dermatitis, and chronic inflammatory diseases whose symptoms appear in other tissues such as intestine and colon, lung and vascular tissue; e.g. abnormal immune reactivity in autoimmune diseases, or undesired immunological reactions associated with therapeutic treatment of diseases as in transplant rejection crises; e.g. immune hypo-reactivity or suppression in diseases such as cancer, and that associated with persistent viral or microbial infection.

A screening assay may comprise the steps of mixing a candidate compound with a solution containing a polypeptide of the present invention to form a mixture, and determining the binding of the compound to the polypeptide; for example, a complex formed between a polypeptide and a compound e.g. may have altered intrinsic biophysical properties that be determined, e.g. may decrease reactivity of a polypeptide with a specific antibody. Alternatively, a complex formed between a polypeptide of the present invention and a compound may have altered binding properties to a calmodulin polypeptide, according to the present invention; where an assay would consist in a first step of mixing a polypeptide with a test compound, as a second step of adding a calmodulin polypeptide to the mixture, and as a third step of measuring effects of the compound on binding of the corresponding substance and calmodulin polypeptides, relative to such binding in the absence of any compound, by e.g. altered intrinsic biophysical properties of the complex of the substance and calmodulin polypeptides.

Thus in another aspect, the present invention provides an assay for screening to identify agonists or antagonists, e.g. including low molecular weight compounds in chemical libraries and natural product libraries, and antisense oligonucleotides, which decrease or enhance the production and or the biological activity of a polypeptide according to the present invention, e.g. a method of identifying an agonist or antagonist, which comprises:
(a) a polypeptide of the present invention or a modified polypeptide fused to a marker polypeptide, according to the present invention;
(b) a host cell supporting an expression system comprising a polypeptide of the present invention or a modified polypeptide fused to a marker polypeptide, according to the present invention;
(c) a host cell supporting an expression system comprising the gene promoter and a recombinant polynucleotide transgene fused to a marker polynucleotide that expresses the fusion polypeptide according to the present invention; or
(d) an antibody to a polypeptide of the present invention or to a modified polypeptide fused to a marker polypeptide, according to the present invention;
e.g. and means for a contact with a candidate compound;
e.g. and means for determining the affect of the candidate compound on any of a), b), c) or
d); e.g. determining whether in the presence of the candidate compound there is a decrease or enhancement in the expression and or the biological activity of a gene according to the present invention; e.g. by comparison of the activity of any of a), b), c) or d) in the presence and in the absence of the candidate compound.

It will be appreciated that in any such screening assay, a), b), c) or d) may comprise a substantial component.

A candidate compound includes any one of the chemical entities as described above, present in e.g. natural or synthetic compound libraries, i.e. systematic collections of chemical entities, for which the affect on any of a), b), c) or d) in an assay for screening according to the present invention is unknown.

An antagonist or agonist is a candidate compound for which an affect on any of a), b), c) or d) has been found in a screening assay or using a method for identifying antagonists or agonists as described above. An antagonist or agonist may decrease or enhance, respectively, the expression and or the biological activity of a gene according to the present invention.

In another aspect the present invention provides an antagonist or an agonist of the expression and or the biological activity of a gene according to the present invention, that is characterized in that said antagonist or agonist can be provided according to the method of screening and identification as described above.

In another aspect the present invention provides an antagonist or agonist as described above that may have immune modulatory activities and may be used as a pharmaceutical intervention in the treatment of diseases, e.g. such as acute and chronic inflammatory diseases, including for example inflammatory skin diseases such as allergic and atopic dermatitis, and chronic inflammatory diseases whose symptoms appear in other tissues such as intestine and colon, lung and vascular tissue; e.g. such as abnormal immune reactivity in autoimmune diseases, or undesired immunological reactions associated with therapeutic treatment of diseases as in transplant rejection crises; e.g. immune hypo-reactivity or suppression in diseases such as cancer, and that associated with persistent viral or microbial infection; e.g.

An antagonist or an agonist of the expression and or the biological activity of a gene according to the present invention, that is characterized in that said antagonist or agonist can be provided by the following method steps:
A) contacting
   (a) a polypeptide according to TABLE 1;
   (b) a host cell supporting an expression system comprising a polypeptide according to TABLE 1;
   (c) a host cell supporting an expression system comprising the gene promoter and a recombinant polynucleotide transgene fused to a marker polynucleotide that expresses the fusion polypeptide according to TABLE 1; or
   (d) an antibody to a polypeptide according to TABLE 1.
with a candidate compound,
B) determining the effect of the candidate compound on any of a), b), c) or d);
C) choosing an agonist or antagonist determined in step B).

An antagonist or agonist according to the present invention may be useful as a pharmaceutical intervention in treatment of a disease.

In another aspect the present invention provides an antagonist or an agonist according to the present invention for use as a pharmaceutical.

For that use, several approaches are available. If the activity of a gene of the present invention in dendritic cells, or cells of the immune system of similar biological function, according to the present invention, is present in excess in a disease, one approach comprises administering to a subject an antagonist according to the present invention, e.g. in combination with a pharmaceutically acceptable excipient, in an amount effective to inhibit the expression or activity of a gene of the present invention, i.e. of a polypeptide of a gene, or by affecting a second activity dependent upon the activity of a gene of the present invention, and thereby alleviating the abnormal condition associated with a disease. If the activity of a gene in dendritic cells, or cells of the immune system of similar biological function, according to the present invention, is present in paucity in a disease, one approach comprises administering to a subject an agonist according to the present invention, e.g. in combination with a pharmaceutically acceptable excipient, in an amount effective to enhance the expression or activity of a gene of the present invention, i.e. of a polypeptide of a gene, or by affecting a second activity dependent upon the activity of a gene, and thereby alleviating the abnormal condition associated with a disease.

In another aspect the present invention provides an antagonist or agonist of the expression or activity of a gene of the present invention in dendritic cells, or cells of the immune system of similar biological function, according to the present invention, that alters the biological activity of those cells in interacting with, i.e. having a biological affect upon, other types of cells or similar cells, either treated or untreated by, and/or affected or unaffected by exposure to, the antagonist or agonist.

For example, T cells may be stimulated or anergized by interaction with dendritic cells that have been altered by treatment with an antagonist or agonist according to the present invention, by administering the antagonist or agonist to a subject and thus exposing both said dendritic cells and T cells of the subject to the agent.

In yet another approach expression of a gene of the present invention in dendritic cells, or cells of the immune system of similar biological function, according to the present invention, may be inhibited using expression blocking techniques. Such known techniques involve the use of antisense oligonucleotides, either internally generated or separately administered, e.g. according to O'Connor, J. Neurochem (1991) 56:560, in Oligodeoxynucleotides as Antisense Inhibitors of Gene Expression, CRC Press, Boca Raton, Fla. (1988). Alternatively, oligomers, e.g. oligonucleotides that form triple helices with a gene according to the present invention, may be supplied, e.g. according to Lee et al., Nuc Acids Res (1979) 6:3073; Cooney et al., Science (1988) 241:456; Dervan et al., Science (1991) 251:1360. Such oligomers may be administered per se or may be expressed in vivo.

For treatment of abnormal conditions in diseases related to a paucity in expression of a gene of the present invention in dendritic cells, or cells of the immune system of similar biological function, according to the present invention, gene therapy may be used to effect the endogenous expression of a gene as a transgene according to the present invention by the relevant cells in a subject who is thus treated. For example, a gene according to the present invention may be engineered for expression in a replication-defective retroviral vector; a retroviral expression construct obtained may be isolated and introduced into a packaging cell transduced with said construct, thus producing infectious viral particles comprising a principle of transfer of an expression system of a gene according to the invention. Said viral particles may be isolated and directly administered to a subject as treatment; or first administered to isolated cells of a subject for establishment of the expression system, then said treated isolated cells re-introduced into the subject. Alternatively, gene therapy may be used to establish an expression system comprised of a form of a gene whose activity antagonizes that of an endogenous gene, e.g. comprised of a transdominant inhibitor form of a gene, as a treatment of abnormal conditions in diseases related to an excess in expression of an endogenous gene in dendritic cells, or cells of the immune system of similar biological function, according to the present invention. For an overview of gene therapy, see e.g. Chapter 20, Gene Therapy and other Molecular Genetic-based Therapeutic Approaches, (and references cited therein) in Human Molecular Genetics, T Strachan and A P Read, BIOS Scientific Publishers Ltd (1996).

In a further aspect the present invention provides a method of treating abnormal conditions related to both an excess of and insufficient level of expression of a gene according to the present invention; or related to both an excess of and insufficient activity of a polypeptide according to to the present invention, comprising administering a therapeutically effective amount of an agonist or antagonist according to the present invention to a subject in need of said treating; e.g. including a method of treating abnormal conditions in diseases related to either an excess or a paucity of the expression or activity, or abnormal activity, of a gene in dendritic cells, or cells of the immune system of similar biological function, according to the present invention, by any of the approaches described above. Such diseases include e.g. acute and chronic inflammatory diseases, including for example inflammatory skin diseases such as allergic and atopic dermatitis, and chronic inflammatory diseases whose symptoms appear in other tissues such as intestine and colon, lung and vascular tissue; e.g. such as abnormal immune reactivity in autoimmune diseases, or undesired immunological reactions associated with therapeutic treatment of diseases as in transplant rejection crises; e.g. immune hypo-reactivity or suppression in diseases such as cancer, and that associated with persistent viral or microbial infection.

In a further aspect the present invention provides a method of treating acute and chronic inflammatory diseases, including for example inflammatory skin diseases such as allergic and atopic dermatitis, and chronic inflammatory diseases whose symptoms appear in other tissues such as intestine and colon, lung and vascular tissue; e.g. such as abnormal immune reactivity in autoimmune diseases, or undesired immunological reactions associated with therapeutic treatment of diseases as in transplant rejection crises; e.g. immune hypo-reactivity or suppression in diseases such as cancer, and that associated with persistent viral or microbial infection; comprising administering a therapeutically effective amount of an agonist or antagonist preferably an antagonist, according to the present invention to a subject in need of such treatment.

In a further aspect the present invention provides a pharmaceutical composition comprising a pharmaceutically effective amount of an agonist or an antagonist according to the present invention; e.g. an agonist or an antagonist of the expression or activity of a gene of the present invention, in combination with pharmaceutically acceptable carrier(s)/excipient(s).

In a further aspect the present invention provides a method of treating diseases as described above by administering an effective amount of an agonist or antagonist in the form of a pharmaceutical composition to a subject in need of such treatment.

Preferred forms of systemic administration of a pharmaceutical composition according to the present invention include injection, typically by intravenous injection. Other injection routes, such as subcutaneous, intramuscular, or intraperitoneal, may be used. Alternative means for systemic administration include transmucosal and transdermal administration, e.g. using penetrants such as bile salts or fusidic acids or other detergents, or solvents such as dimethyl sulfoxide or alcohols. In addition, if properly formulated in enteric or encapsulated formulations, oral administration may also be possible. Administration of a composition according to the present invention may also be topical and/or localized, e.g. in the form of salves, pastes, gels, etc.

The dosage range required may depend on the choice of an agonist or antagonist, the route of administration, the nature of the pharmaceutical composition, the dinical condition of a subject to be treated, and the judgment of the attending practitioner. Suitable dosages, however, may be in the range of 0.1 to 100 μg of an agonist or antagonist per kg body weight of a subject. Variations in the needed dosage, however, may be expected in view of the variety of compounds available and the differing efficiencies of various routes of administration. For example, oral administration would be expected to require higher dosages than administration by intravenous injection. Variations in these dosage levels may be adjusted, e.g. according to a standard empirical routine for optimization.

Polypeptides and polynucleotides used in treatment may also be generated endogenously in a subject, e.g. in need of such treatment, in treatment modalities often referred to as “gene therapy”, e.g. as described above. Thus, for example, cells from a subject may be engineered ex vivo with a polynucleotide, such as DNA or RNA, to encode a polypeptide or polynucleotide of the present invention, e.g. by use of a retroviral plasmid vector as a principle of transfer as described above. Engineered cells may be introduced into the subject, e.g. in need of such treatment.

In another aspect the present invention provides a gene of the present invention expressed in DCs of the immune system, e.g. comprising the polynucleotide sequence as given in TABLE 1, and encoding a protein of the corresponding polypeptide sequence, whose expression in DCs is regulated by inflammatory stimuli, as a specific molecular target in DCs for therapeutic intervention in diseases in which immunological reactions are a primary component of etiology, progression and/or exacerbating symptoms associated with the disease.

Such diseases include e.g. diseases as described above.

### DESCRIPTION OF THE FIGURES

FIG. 1 is an analysis of the expression of the BASP1 gene in dendritic cells and other cell types of the immune system demonstrating the relevance of the induced expression in dendritic cells stimulated by specific conditions of cellular activation that relate to the activity of dendritic cells within the immune system. BASP1 expression is stimulated in monocyte-derived dendritic cells by stimulation by phagocytosis of zymosan yeast particles and crosslinking of the CD40 protein expressed on the surface of the cells, as shown by comparison of the first two lanes to the left, in which the level of expression is measured by the relative amount of DNA of the full length coding sequences of BASP1 obtained by repeated cycles of DNA synthesis from cDNA from mRNA from unstimulated and stimulated cells (upper panel); the difference observed is specific for the expression of the BASP1 gene, shown by comparison to that of a second gene, β-Actin (lower panel). The upper panel to the right shows results of Northern blot hybridization analysis of BASP1 mRNA expression in many types of cell samples; the lower panel to the right is an image of the same blot used for hybridization analysis (dye staining of cellular RNA transferred onto the blot, revealing that equivalent amounts of total RNA from the different cell samples have been analyzed, and equivalent quality of the prepared RNAs, judged by the relative intactness of the ribosomal RNA [rRNA] species that are most apparent from the staining in the image). Unstimulated and stimulated (treatment with the lectin phytohemagglutinin [PHA]) T cells reveal little or no detectable BASP1 expression by hybridization (the first two lanes to the left of the upper right panel); stimulated B cells (treatment with pokeweed mitogen and IL-4) reveal slight expression (lane 3); unstimulated monocytes reveal little or no expression, whereas stimulated monocytes (treated with GM-CSF) reveal detectable but relatively low expression (lanes 4 and 5, respectively); umbilical cord blood dendrftic cells cultured in the presence of Flt-3 Ligand and IL-3 reveal little or no expression, whereas detectable expression is induced by stimulation of such cells with LPS (lanes 6 and 7, respectively); umbilical cord blood dendritic cells cultured in the presence of GM-CSF, TNFα and TGFβ reveal detectable expression, that is significantly induced by treatment of cells with LPS, and somewhat induced by treatment by crosslinking of the CD40 protein expressed on the surface of the cells with anti-CD40 antibody (lanes 8, 9 and 10, respectively); umbilical cord blood dendritic cells cultured in the presence of GM-CSF and IL-4 reveal little expression, that is strongly induced by treatment of cells with LPS (lanes 11 and 12, respectively).

FIG. 2 is an analysis of the expression of a GID2 gene (polynucleotide) in DC and other cell types of the immune system demonstrating the relevance of the induced expression in DC stimulated by specific conditions of cellular activation that relate to the activity of DC within the immune system. GID2.1 and GID2.2 expression is stimulated in monocyte-derived DC by stimulation by phagocytosis of zymosan yeast particles and crosslinking of the CD40 protein expressed on the surface of the cells, as shown by comparison of the first two lanes to the left, in which the level of expression is determined by the relative amount of DNA of the full length coding sequences (SEQ ID NO:3 and SEQ ID NO:5) obtained by repeated cycles of DNA synthesis from cDNA from mRNA from unstimulated and stimulated cells (upper panel). The difference observed is specific for the expression of the GID2 polynucleotide, shown by comparison to that of a second gene, β-Actin (lower panel). The panel to the right shows results of Northern blot hybridization analysis of GID2 mRNA expression in many types of cell samples: unstimulated and stimulated (treatment with the lectin phytohemagglutinin [PHA]) T cells reveal little GID2 expression (the first two lanes to the left of the panel); unstimulated and LPS-stimulated monocytes reveal little expression (lanes 3 and 4, respectively); unstimulated MoDC reveal slight expression, that is significantly increased by stimulation with LPS (lanes 5 and 6, respectively); unstimulated umbilical cord blood DC reveal appreciable levels of expression, that is further increased by LPS stimulated (lanes 7 and 8, respectively); little or no expression is detected in human umbilical cord vein endothelial cells (HUVEC) stimulated by phorbol myristate (PMA), by TNFα, or stimulated (lanes 9, 10 and 11, respectively).

FIG. 3 is an analysis of the expression of the GID3 gene in DC and monocytes, demonstrating the relevance of the induced expression in these two types of cells in response to immune-inflammatory stimuli. GID3 expression is stimulated in monocyte-derived DC by stimulation by phagocytosis of zymosan yeast particles and crosslinking of the CD40 protein expressed on the surface of the cells, as shown by comparison of the first two lanes the the left, in which the level fo expression is determined by the relative amount of DNA of the full length coding sequences (SEQ ID NO:8) obtained by repeated cycles of DNA synthesis from cDNA (from mRNA) from unstimulated and stimulated cells (upper panel); the difference observed is specific for the expression of the GID3 gene, shown by comparison to that of a second gene, β-Actin (lower panel). The panel to the right shows results of Northern blot hybridization analysis of GID3 mRNA expression in several different cell samples: unstimulated monocytes (the first left lane in the panel to the right) reveal no GID3 expression, whereas stimulation of monocytes by interferon-γ (IFN-γ), bacterial lipopolysaccharide (LPS) or granulocyte-macrophage colony stimulating factor (GM-CSF) significantly elevate GID3 expression (lanes 2, 3 and 4, respectively). Monocyte-derived DC, either unstimulated (lanes 5 and 10) or stimulated by CD40 cross-linking (lane 6), express low or undetectable levels of GID3, however expression is significantly elevated in these cells by a combination of phagocytosis/CD40 cross-linking (lane 7) (confirming results in the panel to the left) or a combination of the inflammatory cytokines TNFα/IL-1β (lane 11); unstimulated umbilical cord blood DC reveal a low level of expression (lane 8), that is increased by CD40 cross-linking (lane 9).

FIG. 4 shows the sequence homology of the GID4 protein sequence to the consensus DUF221 sequence. FIG. 4 is an alignment of the GID4 polypeptide sequences from position 348-809 of SEQ ID NO:12 with the DUF221 PFAM signature, obtained by search of the PFAM Database Release 6 using the HMMR software (Washington University, St. Louis).

FIG. 5 shows a conserved structural organization, as defined by the relative position of the DUF221 domain within the GID4 protein, and the distribution of potential transmembrane domains and N-glycosylation sites. In FIG. 5 the structure arrangement of the GID4 protein sequence SEQ ID NO:12 is compared with respect to the position of the DUF221 domain, the potential transmembrane domains, and the potential N-glycosylation sites, with the corresponding arrangement of these elements in the sequences of the homologous human proteins KIAA0792 (TREMBL:O94886) and 93.3/93.8 KDA (TREMBL:Q9P1W3 and Q9P1W1), and the D. melanogaster protein CG11210 (TREMBL:Q9V364). The method of analysis is described in Example 1.

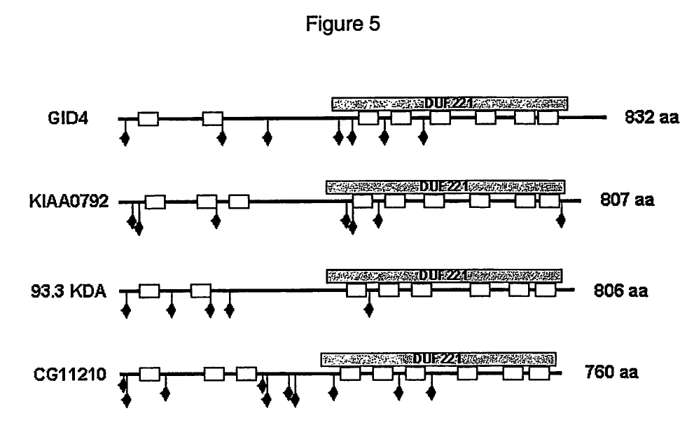

FIG. 6 shows the induction of GID4 gene expression in response to stimulation of DC. In FIG. 6 an analysis of the expression of the GID4 gene in DC and other types of cells of the immune system, demonstrating the specificity of induced GID4 expression in DC, and the relevance of induced expression to inflammatory stimuli is given. GID4 expression is stimulated in monocyte-derived DC by stimulation by phagocytosis of zymosan yeast particles and crosslinking of the CD40 protein expressed on the surface of the cells, as shown by comparison of the first two lanes in the panels to the left, in which the level of expression is measured by the relative amount of DNA of the full length coding sequences (SEQ ID NO:12) obtained by repeated cycles of DNA synthesis from cDNA (from mRNA) from unstimulated and stimulated cells (upper panel); the difference observed is specific for the expression of the GID4 gene, shown by comparison to that of a second gene, β-Actin (lower panel). The panel to the right shows results of Northern blot hybridization analysis of GID4 mRNA expression in several different cell samples: unstimulated T cells cultured with IL-2 (the first left lane in the panel to the right) reveal no GID4 expression, whereas GID4 expression in stimulated T cells (treatment with the lectin phytohemagglutinin, lane 2) and stimulatedB cells (treatment with pokeweed mitogen and IL-4, lane 3) is detectable, albeit at very low levels. GID4 is expressed at low levels in unstimulated monocyte-derived DC (MoDC), significantly increased by stimulation with bacterial lipopolysaccharide (LPS)(lanes 4 and 5, respectively). Expression at levels comparable to that in unstimulated MoDC is also seen in DC obtained by culturing umbilical cord blood precursor cells with GM-CSF/TNFα/TGFβ (lane 7) or GM-CSF/TNFα/IL-4 (lanes 8 and 10); somewhat higher levels of GID4 are seen in LPS-stimulated DC derived from precursor cells cultivated with Flt-3 ligand and IL-3 (lane 6); LPS stimulation for 6 hr and 18 hr (lanes 9 and 11, respectively) of DC derived from precursor cells cultivated with GM-CSF/TNFα/IL-4 reveals a marked increase in GID4 expression with longer stimulation. Methods are detailed in Example 2.

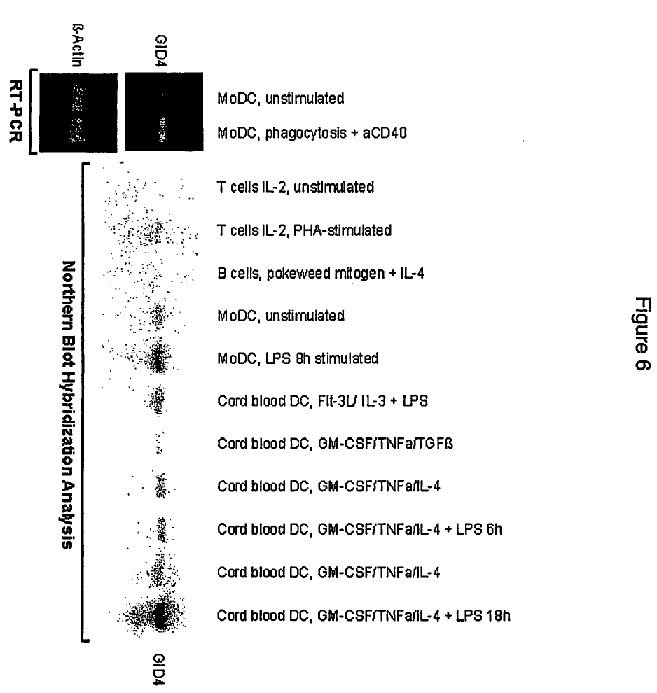

FIG. 7 shows IL-8 secretion measured by specific ELISA following LPS-stimulation of cells that ectopically express BASP 1 (U937_BASP) compared to vector control-containing cells (U937_vector).

FIG. 8 shows the results of a FACS analysis of antigen-presenting cells that ectopically express an epitope-tagged GID2.2 polynucleotide compared to vector control-containing cells, confirming expression of GID2.2 on the surface of AB4 cells.

FIG. 9 shows the antigen-dependent stimulation of a T cell clone (TCC) by co-cultivation with antigen-presenting cells (AB4) that ectopically express GID2.2 polypeptide (AB4-GID2.2) compared with control vector-containing AB4 cells. In the analysis, the ratio of antigen-presenting cells to TCC is varied as indicated. As antigen-negative control a 1:1 mixture of 50000 AB4 cells and 50000 TCC is also shown.

### EXAMPLES

### Example 1

Isolation of BASP1, GID2, GID3 and GID4 Polynucleotides from Dendritic Cells and Sequence Analysis

cDNA clones comprising BASP1, GID2, GID3 and GID4 sequences of varying 5′ lengths are isolated from cDNA libraries prepared from dendritic cell mRNA by hybridization with a small (200-bp) BASP1, GID2, GID3 and GID4 probe derived from subtraction of cDNA prepared from stimulated (zymosan and anti-CD40 antibody) and unstimulated dendritic cell mRNA, and comparison of sequences of the cDNA clones with the GenBank database using the BLASTN algorithm identified the sequences as >98% identical to that of the entry for NAP22 (another name for BASP1 ), AF039656 or using the FRAMES software of the GCG SeqWeb (Wisconsin Package) in the case of the GID probes. For confirmation of the recombinant cDNA phagemide clones, and the predicted open reading frames, oligonucleotide primers flanking the coding sequences are used to independently obtain clones directly from dendritic cell cDNA by repeated cycles of synthesis using a thermostable DNA polymerase (KlenTaq Polymerase, Clontech), and the sequences of three different clones thus derived yields the consensus
SEQ ID NO: 1 encoding a BASP1 polypeptide;
SEQ ID NO:3 encoding a GID2.1 polypeptide;
SEQ ID NO:5 encoding a GID2.2 polypeptide; (the polypeptide sequences SEQ ID NO:4 and SEQ ID NO:6 correspond to said encoding polynucleotide sequences);
SEQ ID NO:8 encoding a GID3 polypeptide; (the polypeptide sequence GID3 SEQ ID NO:9 corresponds to the polynucleotide sequence SEQ ID NO:8);
consensus SEQ ID NO:12 encoding a GID4 polypeptide; (the polypeptide sequence GID4 SEQ ID NO:12 corresponds to the polynucleotide sequence SEQ ID NO:12).

It was observed that inclusion of 5% dimethyl sulfoxide, which lowers the effective melting temperature of polynucleotide duplex structures, is necessary for reproducible synthesis with KlenTaq polymerase, most likely due to the strikingly GC-rich sequence content of SEQ ID NO: 1 and related sequences such as AF039656. Since
i) the translation of SEQ ID NO: 1 agrees 100% with the BASP1 polypeptide sequence of the SwissProt database entry P80723, that was derived from protein sequencing, i.e. independent of any in vitro cDNA or DNA synthetic steps; and
ii) sequences of three BASP1 DNA clones synthesized as described above from dendritic cell cDNA, using KlenTaq polymerase in the presence of 5% dimethyl sulfoxide, agree with the consequence SEQ ID NO: 1;
we conclude that SEQ ID NO: 1 reflects the correct sequence of BASP1 expressed as mRNA in dendritic cells and that the sequences in the GenBank database entries AF039656 and NM_{—}006317 (described in the first section of the invention) are incorrect.
   Monocyte-Derived Dendritic Cells

Human monocytes obtained by countercurrent elutriation are seeded into Costar Swell plates (Costar, Cambridge, Mass.) at 8×10⁵ cells/ml in RPMI1640 medium (Gibco-BRL, Rockville, Md.) containing 10% fetal calf serum and supplemented with GM-CSF (Novartis AG, Basel, Switzerland)(300 U/ml) and rIL4 (R&D Systems)(200 U/ml). Cultures are fed on day 3 by exchanging half of the medium with fresh medium that had been suplemented with GM-CSF and rIL4 to achieve the final concentrations indicated above.

RNA Isolation, cDNA and DNA Synthesis, and Construction of Recombinant Plasmid Clones

RNA is isolated using the Trizol reagent (Gibco-BRL, Rockville Md.). cDNA is synthesized using the combination of SuperScript (RNaseH activity destroyed by mutation), Moloney leukemia virus reverse transcriptase, RNaseH, DNA polymerase and DNA ligase purified from E. coli (Gibco-BRL, Rockville Md.); for cDNA libraries, the cDNA is modified by ligation of linker-adaptors, specific digestion with the restriction enzymes SalI and NotI according to design, and ligation into the pSPORT1 plasmid vector DNA (Gibco-BRL, Rockville Md.) as a SalI-NotI insertion.

For independent derivation of DNA directly from cDNA template, repeated cycles of DNA synthesis are performed with KlenTaq thermostable DNA polymerase (containing a 3′-5′ exonuclease proofreading activity to lower the frequency of polymerase error)(Clontech) in the presence of 5% (v/v) dimethyl sulfoxide, as extension products of the following synthetic oligonucleotides

for BASP1 DNA:
5′-CGAGCCGAACTCCAAGATGG-3′
and
5′-GGTCCTTGTCACTCTTTCACG-3′
for GID2.1 DNA:
5′-CCACGCATGACGGTGCATG-3′
and
5′-GGAAACTCAGGGTATTCCCAC-3′
for GID2.2. DNA:
5′-ATGGGCGCCCTCAGGCCCAC-3′
and
5-TCACCGTTTTCGAAGCCTCTTC-3′
for GID3 and GID4:
5′-GGCGCCATGAGTGGCGGCGG-3′
and
5′-GGTGCTGGGAGGTCAGATGTCG-3′
(all from Genset, Paris),

followed by end repair with the Klenow fragment of DNA polymerase (New England Biolabs), and ligation into EcoRI-digested pSPORT1 plasmid vector DNA whose ends had been blunted with the same enzyme.
Alignment of DNA Sequences and Analysis of Databases

The algorithm ClustalW is used in alignment of DNA sequences; the BLASTN algorithm is used to identify similar sequences present in the GenBank-EMBL DNA sequence databases; the BLASTX algorithm is used to identify sequences in the SwissProt protein sequence database similar to polypeptide sequences encoded by DNA.

Transmembrane helix prediction is performed according to G. von Heijne, J. Mol. Biol. 225 [1992] 487-494. Polypeptide motifs are obtained by Motif search of the PROSITE Dictionary of Protein Sites and Patterns. HMMPFAM are obtained by searching the HMM database HMMER 2.1.1 [December 1998], Washington University School of Medicine.

### Example 2

Expression of BASP1, GID2, GID3 and GID4 mRNA in Dendritic Cells and Other Cells of the Immune System

Methods

Monocytes, T cells and B cells are isolated from peripheral blood by enrichment by centrifugal elutriation; cord blood dendritic cells are obtained as precursor cells from umbilical cord blood and cultivated in the presence of factors that promote their differentiation into immature dendritic cells. RNA isolation, cDNA synthesis, and repeated cycles of DNA synthesis using a thermostable DNA polymerase, are performed as described above. Separation of RNA by electrophoresis, blotting and hybridization with a synthetic oligonucleotide probe (Northern hybridization analysis) are performed as described in Sambrook et al., Molecular Cloning: A Laboratory Manual, 2nd Ed., Cold Spring Harbor Laboratory Press, Cold Spring Harbor, N.Y. (1989).

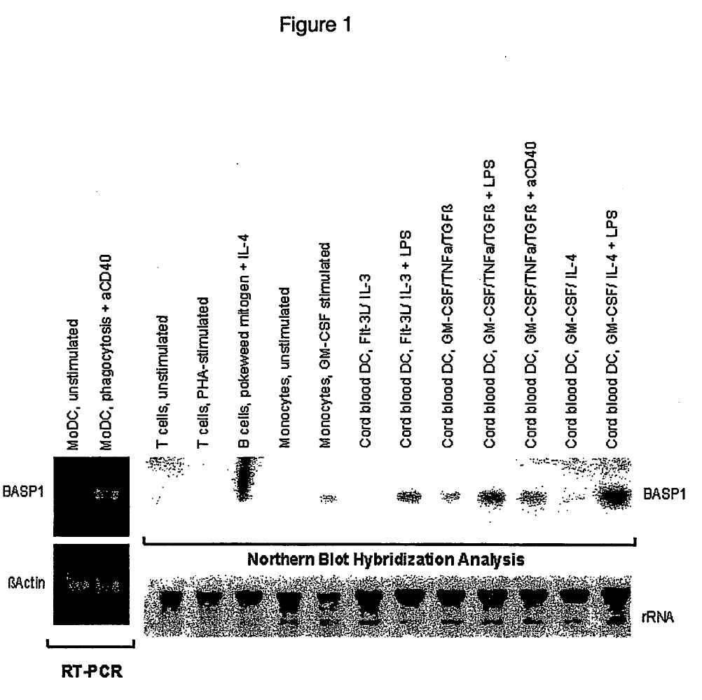

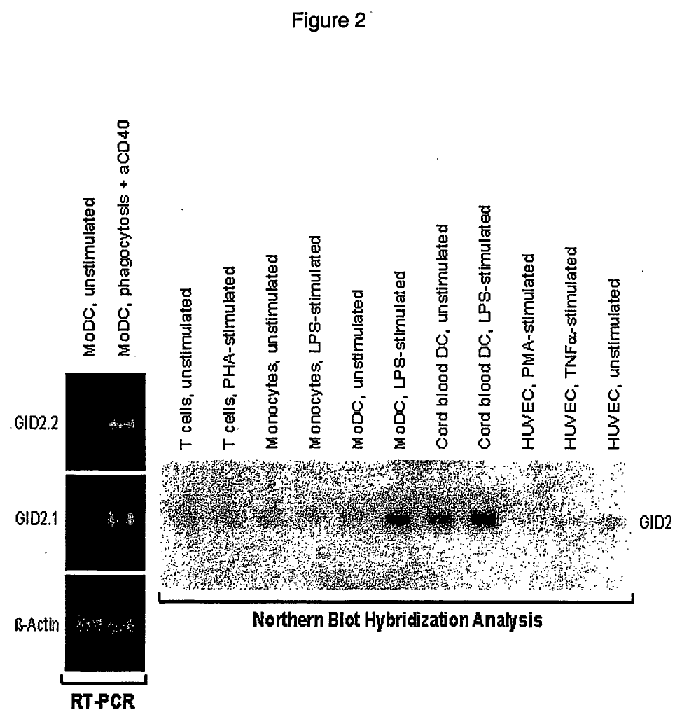

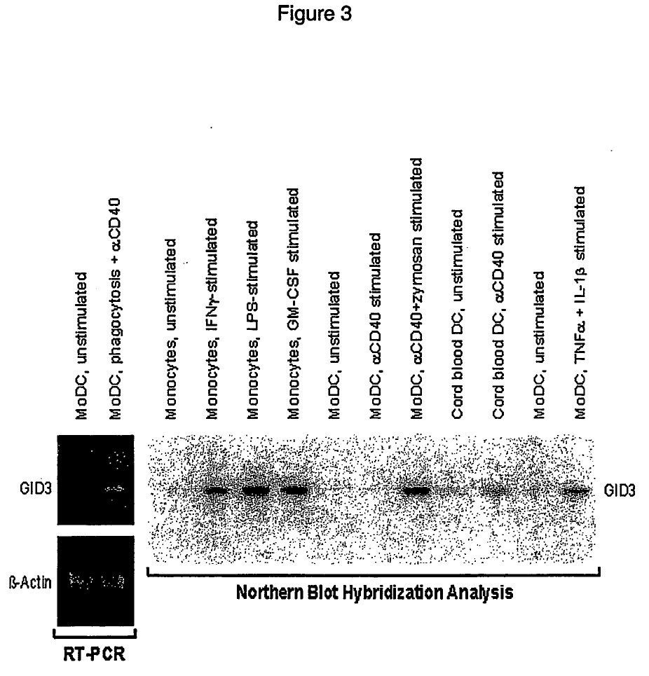

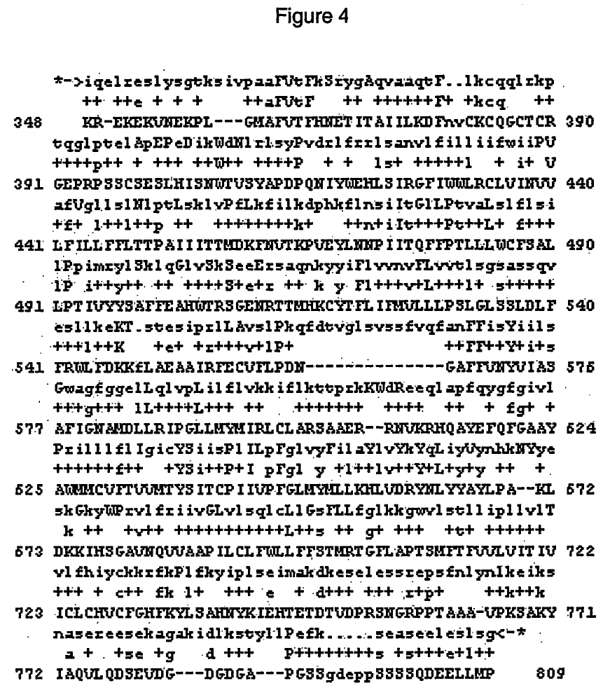

As shown in the FIGS. 1, 2, 3 and 4, expression of appropriate mRNA in dendritic cells and other types of cells of the immune system, unstimulated or stimulated by relevant immune activation signals, may be analyzed by either 1) RT-PCR of the full-length BASP1 coding polynucleotides (using the primers described above), and normalizing that expression for that of a uniformly expressed polynucleotide sequence such as β-actin mRNA; or 2) Northern hybridization analysis.

Such an analysis indicates the following for the different substances:

Expression of BASP1 mRNA according to the Invention is significantly enhanced in various types of dendritic cells, and dendritic cells matured by cultivation in different specific growth factors and cytokines, by the relevant stimuli. Furthermore, much lower levels of expression of BASP1 mRNA are detected in stimulated or unstimulated T cells, B cells or monocytes; of the latter three types of immune cells, monocytes stimulated with GM-CSF do show detectable BASP1 mRNA expression, but not as pronounced as in stimulated dendritc cells.

Expression of GID2 mRNA is significantly enhanced in various types of DC, and DC matured by cultivation in different specific growth factors and cytokines, by the relevant stimuli. Furthermore, much lower levels of expression of GID2 mRNA are detected in stimulated or unstimulated T cells, monocytes or endothelial cells.

Expression of GID3 mRNA is significantly enhanced in monocytes, monocyte-derived DC, and DC matured from cord blood precursor cells, by the specific stimuli relevant to immune system activation, e.g. inflammatory stimuli.

Expression of GID4 mRNA is significantly enhanced in monocyte-derived DC by a phagocytic stimulation combined with CD40 cross-linking, and by bacterial lipopolysaccharide (LPS) stimulation in monocyte-derived DC, and in DC matured from cord blood precursor cells by cultivation with granulocyte-macrophage colony stimulating factor (GM-CSF), tumor necrosis factor-α (TNFα) and interleukin-4 (IL-4). In contrast, only detectable or no GID4 expression is seen in unstimulated or stimulated T cells, or in stimulated B cells. Thus, GID4 expression is restricted, relative to T and B cells, to DC, and the induced GID4 expression in DC by specific inflammatory stimuli demonstrates the relevance to activation of the immune system.

### Example 3

Production of BASP1, GID2, GID3 and GID4 Polypeptides and Recombinant Polypeptides

Based upon experience and commonly used methods in the production of recombinant proteins, expression vectors can be prepared for the production of the BASP1, GID2, GID3 and GID4 polypeptide and variants thereof. The expressed transgenic polypeptide from stably transfected producer cells may be isolated and purified, and used for the generation of antibodies and functional studies, as well as in screening assays. Recombinant variants of appropriate polypeptides containing N- and C-terminal tag, as well as the unmodified forms of the polypeptide, may be produced. Expression of tagged recombinant protein may be performed by preparing constructs in the FLAG-containing pcDNA vectors obtained commercially (InVitrogen and Sigma, as well as other suppliers). Proteins may be produced in E. coli strains that are also available commercially. For producing large amounts of proteins that a post-translationally modified (e.g. glycosylated), the Baculovirus expression vectors in insect cells may be used, or vectors designed for selection and expression in yeast cells; in both cases, expression vectors and suitable producer cells may be obtained commercially, and are commonly used. Protein in mg amounts is produced, as it is of advantage for immunizations, and necessary for functional studies and screening assays according to the invention.

### Example 4

Generation of Monoclonal Antibodies

Monoclonal antibodies may be obtained by common methods used in mice immunized with the produced BASP1, GID2, GID3 and GID4 protein, where antibodies produced by the resulting hybridoma cell lines may be screened against, and the antibodies purified by affinity chromatography using, the immunizing protein. Tag-containing recombinant BASP1, GID2, GID3 or GID4 protein may also be used to immunize mice to produce antibodies, in which case antibodies produced by the resulting hybridoma cell lines will be screened for epitope-specific antibodies either against the unmodified form of the BASP1, GID2, GID3 or GID4 protein, or against both the tag-containing protein and a second tag-containing protein, such as glutathione-S-transferase (GST)-tag fusion proteins. The antibodies may be used in studies to detect expression of appropriate polypeptides in functional studies, such as inhibiting BASP1, GID2, GID3 or GID4 expression with antisense oligonucleotides, and testing the effects of low molecular weight antagonists and agonists identified in screening assays, described below. Antibodies may also comprise a component of a diagnostic method and kit.

### Example 5

Inhibition of BASP1, GID2, GID3 and GID4 Expression with Antisense Oligonucleotides

The use of synthetic antisense oligonucleotides (ASO) composed of small sequence stretches, preferably 18-20 nucleotides in length, that are complementary to specific sequences of the sense polyribonucleotide (mRNA) strand of a given gene, that when introduced into cells will bind that mRNA producing duplex structures thereby being recognized by double-strand RNase enzymes that subsequently degrade the intact mRNA molecule and abrogate expression of the encoded protein of that gene, is a commonly used method for studies of gene function. For inhibition of BASP1 expression, ASO containing complementary BASP1, GID2, GID3 or GID4 sequences are introduced into dendritic cells, and certain cell lines that express BASP1, GID2, GID3 or GID4 mRNA, by common techniques such as e.g. the use of liposomal agents that are commercially available (e.g. Lipofectamine and Lipofection, GIBCO-BRL). The effects of the ASO are determined by any of several methods, such as RNase protection assays, S1 nuclease assays, PCR, or ELISA assays using an anti-BASP1 monoclonal antibody described above. Several different ASO, each containing sequences complementary to different regions of the BASP1, GID2, GID3 or GID4 mRNA, are tested for activity in the assays described, and the most effective ASO are selected for uses described in subsequent examples below. The specificity of the activity of the ASO are determined by determining the activity of ASO of a related sequence that contains several, preferably 3 to 5, nucleotide substitutions in the assays described, e.g. controlling the activity of a specific ASO by using a “mismatch” ASO in selected assays.

### Example 6

Identifying Activation Markers in Dendritic Cells and Cell Lines Dependent Upon BASP1, GID2, GID3 or GID4 Expression

There are numerous surface membrane-associated proteins expressed in dendritic cells, e.g. MHC Class II proteins, CD40, CD80 and CD86, and soluble mediators secreted by dendritic cells, e.g. G-CSF, IL-12 and IL-1, that are recognized as playing a role in amplification of immunological signals from dendritc cells to T cells and other types of cells of the immune system (heterotypic responses), and to other dendritc cells (homotypic responses). The expression of these types of genes, that may be termed activation markers, in dendritic cells increases dramatically as the cells are activated by immunological signals. The role of BASP1, GID2, GID3 or GID4 expressed in dendritic cells stimulated in different ways, such as with LPS, TNFα, phagocytically, or using appropriate antibodies or ligands to crosslink specific surface proteins, or combinations of such stimuli, upon induction of activation markers in the stimulated cells, is determined by introducing ASO into the cells as described above, followed by stimulation of the cells, and assaying for the expression levels of the activation markers. The specificity of the inhibition is controlled by comparison with cells in which mismatch ASO are introduced as described above, and or using cells that are not treated with ASO in the assay. The expression of the activation markers are determined by any of several methods, such as RNase protection assays, S1 nuclease assays, PCR, ELISA or FACS assays using appropriate antibodies. Activation markers in cell lines described above are also identified by these same methods.

### Example 7

Determination of Interactions of BASP1, GID2, GID3 or GID4 with Other Dendritic Cell Membrane-Associated Proteins

The membrane or subcellular localization of the BASP1, GID2, GID3 or GID4 protein expressed in dendritic cells is examined by fractionating membrane preparations according to density by centrifugation, to determine if the protein is associated with raft structures, as has been demonstrated in neuronal cells (Maekawa, S., J. Biol. Chem. 274 [1999] 21369-21374). In this analysis, proteins contained within various cellular fractions, e.g. cytoplasmic, membrane raft and non-raft membrane fractions, are further fractionated by electrophoresis under denaturing conditions in polyacrylamide gels (SDS gel electrophoresis), blotted onto filters and subjected to Western analysis using the BASP1, GID2, GID3 or GID4 antibody described above. Unstimulated dendritic cells and cells treated with immunological stimuli, such as LPS, TNFα, phagocytically, or using appropriate antibodies or ligands to crosslink specific surface proteins, or combinations of such stimuli, are analyzed to determine if there is altered localization of the BASP1, GID2, GID3 or GID4 protein in response to cellular activation. Other cellular proteins that closely interact physically with BASP1, GID2, GID3 or GID4 in the different fractions, and BASP1, GID2, GID3 or GID4 antibody, with or without treatment with reversible bivalent crosslinking agents (commercially available from Sigma Chemical Company or Pierce), followed by 2D gel separations (isoelectric focusing combined with denaturing SDS gel electrophoresis) and partial microsequencing analysis. The physical interaction of BASP1, GID2, GID3 or GID4 with a second protein expressed in dendritic cells that is identified by these techniques is confirmed by the reverse experiment, in which immunoprecipitation is performed with specific antibody to the second protein, followed by Western blotting analysis using the BASP1, GID2, GID3 or GID4 antibody. Specific antibodies against many known membrane-associated surface proteins are commercially available. The physical interaction with BASP1, GID2, GID3 or GID4 any second protein identified and confirmed in dendritic cells is also confirmed in cell lines described above, when that protein is also shown to be expressed in those cell lines.

### Example 8

Mapping BASP1, GID2, GID3 or GID4 Protein Domains Specifying Protein-Protein Interactions

The DNA expression vector that encodes a modified recombinant BASP1, GID2, GID3 or GID4 protein fused to a tag-polypeptide described above, for example a FLAG epitope, is mutagenized at certain sites by small in-frame insertions that may disrupt domain function within BASP1, GID2, GID3 or GID4. The mutagenized constructs are introduced into cell lines expressed above, and other cellular proteins that physically with the recombinant, mutagenized BASP1, GID2, GID3 or GID4 variant proteins are identified by the methods described above, using an antibody specific for the tag-polypeptide to purify the protein complexes. For example, the domain within the BASP1, GID2, GID3 or GID4 protein that is necessary for the physical interaction with calmodulin, known to occur in neuronal cells (Takasaki, A., J. Biol. Chem. 274 [1999] 11848-11853) should be identified in this way. Confirmation and finer mapping of the BASP1, GID2, GID3 or GID4 protein domain is then performed using the tag-fusion recombinant BASP1, GID2, GID3 or GID4 expression constructs by introducing deletions and point mutations into the BASP1, GID2, GID3 or GID4 protein sequence. In this way, interaction-defective BASP1, GID2, GID3 or GID4 variant proteins are defined, and they may be produced using the expression vectors in producer cells as described above. In addition, a minimal BASP1, GID2, GID3 or GID4 linear polypeptide sequence may be defined, termed an interaction polypeptide sequence, that may be necessary for BASP1, GID2, GID3 or GID4 to interact with the second protein.

### Example 9

Interference with BASP1, GID2, GID3 or GID4 Activities in Dendritic Cells Using Polypeptides

Interaction polypeptide sequences described above are introduced into dendritic cells by using infectious, replication-defective mammalian expression vectors, derived from e.g. suitable scaffolds based upon lentiviruses or spumaviruses, that encode are capable of expressing polypeptides within the infected cells. The effect of the expressed interaction polypeptides in interfering with the interaction of endogenous BASP1, GID2, GID3 or GID4 protein with second proteins as described above, are assayed in proteins extracted from the cells by immunoprecipitation with the anti-BASP1, GID2, GID3 or GID4 antibody described above, followed by analysis of the co-immunoprecipitated proteins, preferably by Western blot analysis using a specific antibody directed against the second protein, as described above. The specificity of a given interaction-interference polypeptide is determined by introducing mutations, i.e. 2 to 4 amino acid substitutions, into the polypeptide in the expression vector, and testing such “mismatch” interaction-interference control polypeptides in the assay. In this way, interaction-interference polypeptides may be identified, and they may be produced in pure form by synthetic methods as conventional.

### Example 10

Identification of Activation Markers in Dendritic Cells and Cell Lines Dependent Upon Specific Physical Interaction of BASP1, GID2, GID3 or GID4 with Second Cellular Proteins

The necessity of specific physical interaction of BASP1, GID2, GID3 or GID4 with second cellular proteins expressed in dendritic cells, and cell lines as described above, that are stimulated in different ways, such as with LPS, TNFα, phagocytically, or using appropriate antibodies or ligands to crosslink specific surface proteins, or combinations of such stimuli, for the induction of activation markers in the stimulated cells, is determined by introducing interaction-inferference polypeptides into the cells as described above using replication-defective infectious viral expression vectors, followed by stimulation of the cells, and assaying for the expression levels of the activation markers. The specificity of the assay is determined by introducing mismatch interaction-interference control polypeptides into the cells that are subjected to the assay.

### Example 11

Phenotypic Properties of Dendritic Cells that are Dependent on BASP1, GID2, GID3 or GID4 Expression and or Specific Physical Interaction of BASP1, GID2, GID3 or GID4 with Second Cellular Proteins

The functional importance of BASP1, GID2, GID3 or GID4 expressed in dendritc cells, in terms of immunologically relevant cellular activities, or properties, are assessed in the following two assays:

i) Dendritic Cell Migration Assays

An early step during activation of immature dendritic cells is the acquisition of migratory capability, that is assessed by e.g. placing the cells into one chamber of a two-chamber system separated by a semipermeable membrane, and measuring the proportion of the cells that migrate through the membrane into the second chamber, or performing microphotography of cells that are suspended in a highly viscous medium that impedes Brownian movement, as collagen-containing gels. The dependence of migratory capacity upon BASP1, GID2, GID3 or GID4 expression is assayed in dendritic cells in which specific ASO directed against BASP1, GID2, GID3 or GID4 mRNA have been introduced, as described above. The specificity of the ASO is determined by including a mismatch ASO in the assay, as described above. The dependence of migratory capacity upon specific physical interaction of BASP1, GID2, GID3 or GID4 with second cellular proteins expressed in dendritic cells is assayed in cells into which specific interaction-interference polypeptides have been introduced, as described above. The specificity of action of the interaction-interference polypeptides is determined by introducing a mismatch interaction-interference control polypeptide into the cells subjected to assay, as described above.

ii) T Cell Activation by Dendritic Cells and Cell Lines

As dendritic cells mature in response to activation, they become increasingly more potent in their ability to activate T cells to replicate by presentation of antigen. This is referred to as the T cell activation capacity of dendritic cells, and it is assayed by mixing a series of decreasing ratios of dendritic cells (that are pre-exposed to a suitable antigen for presentation) to T cells together in culture medium, e.g. 1:5 to 1:25 to 1:100 to 1:300 to 1:1000 etc.; incubating them for typically 5 to 7 days, during which time the dendritic cells process the antigen internally and present the epitopes from their surface in complexes with MHC class 11 proteins, that in turn form complexes with the T cell receptor surface proteins, among other cell-cell communication signals comprising the process of activation; then determining the degree of T cell replication stimulated by the activation process, e.g. by determining the incorporation of radioactive subtrates for DNA synthesis in the replicating T cells. For example, the strength of activation capacity of dendritic cells, or potency, is expressed as the number of T cells per dendritc cell at which the highest degree of T cell replication is stimulated; e.g. the greater the number of T cells that may be stimulated to replicate by a single dendritc cell represents a correspondingly higher activation capacity of that dendritic cell. The dependence of such activation capacity upon BASP1, GID2, GID3 or GID4 expression in dendritic cells is assayed in cells into which specific ASO directed against BASP1, GID2, GID3 or GID4 mRNA have been introduced, as described above. The specificity of the ASO is determined by including a mismatch ASO in the assay, as described above. The dependence upon specific physical interaction of BASP1, GID2, GID3 or GID4 with second cellular proteins expressed in dendritc cells is assayed in cells into which specific interaction-interference polypeptides have been introduced, as described above. The specificity of action of the interaction-interference polypeptides is determined by introducing a mismatch interaction-interference control polypeptide into the cells subjected to assay, as described above.

### Example 12

Recombinant Reporter Gene Vectors Containing BASP1, GID2, GID3 or GID4 Promoter Sequences and Expression in Dendritic Cells and Cell Lines

Transcriptional regulation of mRNA synthesis is typically exerted through chromosomal DNA sequences present directly upstream of the position of mRNA synthesis initiation (i.e. the chromosomal sequences adjacent to the template sequences, flanking the side of initiation), that is termed the gene promoter, and small DNA elements referred to as transcriptional enhancers that may lie distal to the promoter sequences. The chromosomal DNA sequences that would potentially comprise the BASP1, GID2, GID3 or GID4 gene promoter are amplified by PCR, e.g. amplified DNA fragments of lengths varying from 100 to 200 to 500 to 1000 bp, and cloned within a recombinant mammalian expression vector capable of expressing a marker protein that may be easily detected, such as luciferase, whereby when the recombinant expression vector is introduced into mammalian cells the expression of the marker is dependent upon the promoter activity of the inserted amplified DNA fragment. In this way i) the BASP1, GID2, GID3 or GID4 promoter may be functionally defined and grossly localized, and ii) the activity of complex cellular factors, and events that control the activity of those factors, upon which enhanced BASP1, GID2, GID3 or GID4 mRNA expression depends, e.g. such as occurs during dendritic cell activation, may be conveniently and quantitatively monitored by the amount of the marker protein expressed in the cells into which the recombinant expression vector has been introduced. The recombinant expression vector is introduced into dendritic cells BASP1, GID2, GID3 or GID4 promoter is regulated. The degree to which the level of marker expression accurately reflects the activity of the BASP1, GID2, GID3 or GID4 promoter in the recombinant expression vector in an assay designed to measure such effects, as opposed to other unrelated effects, is assessed by including a second marker in a second recombination vector subject to transcriptional control by a second promoter whose activity is unaffected by changes in cellular events occurring during an assay; and the first marker in the second recombinant vector subject to transcriptional control by the said second promoter.

### Example 13

Screening Assays for Low Molecular Weight Agonists or Antagonists of BASP1, GID2, GID3 or GID4 Expression or Activity

Several types of assays are suited for high throughput screening of libraries of small molecular weight compounds, e.g. collections of natural products and combinatorial chemistry libraries, e.g. antisense oligonucleotides, peptides, antibodies and mimetics. Antagonists or agonists of BASP1, GID2, GID3 or GID4 expression, or of the functional activity of the BASP1, GID2, GID3 or GID4 protein, are screened according to the following assays:

i) Cellular Screening Assays for Antagonists or Agonists of BASP1, GID2, GID3 or GID4 Expression

The recombinant expression vector containing a marker protein, whose expression is subject to the BASP1, GID2, GID3 or GID4 promoter, as described above, is introduced into a cell line in which that promoter is shown to be induced by suitable cellular stimulation, as described above. In screening assays, the cells containing the expression vector are contacted with substances present in the libraries, then they are treated under conditions shown to activate the BASP1, GID2, GID3 or GID4 promoter, and compounds screened that result in either higher marker expression (potential agonists), or lower marker expression (potential antagonists), relative to cells that are not exposed to any substance, are selected.

ii) Cellular Screening Assays for Antagonists or Agonists of BASP1, GID2. GID3 or GID4 Activity

Cellular activation markers in cell lines whose expression upon stimulation is dependent upon BASP1, GID2, GID3 or GID4 expression, and or upon specific physical interaction of BASP1, GID2, GID3 or GID4 with second cellular proteins, as described above, are used as indicators of BASP1 cellular activity. An indicator is selected such that its expression may be conveniently and quantitatively detected in cellular assays, such as using antibodies specific for the indicator protein coupled with second signal-generating systems dependent upon bound antibody, e.g. color or fluorescence-generating reactions, e.g. proximity fluorescence or other detectable energy transfer methods. In screening assays, the cells are contacted with substances present in the libraries, then they are treated under conditions shown to activate BASP1, GID2, GID3 or GID4 expression, and compounds screened that result in either activation marker (indicator) expression (potential agonists), or lower activation marker expression (potential antagonists), relative to cells that are not exposed to any substance, are selected.

iii) Screening Assays for Compounds That Specifically Bind BASP1, GID2, GID3 or GID4 Protein

BASP1, GID2, GID3 or GID4 protein isolated and produced, described above, is contacted with substances present in the libraries in an assay such that a substance bound with protein may be discrimated from free (unbound) substance and protein. Substances bound to protein, for example, may alter intrinsic biophysical properties of that protein that may thereby be detected; or a substance may be linked to an indicator molecule of which a biophysical property may be altered when the linked substance binds to, and therefore brings into the proximity to the indicator, a protein molecule; or for example in types of competition assays, in which an antibody or a mimetic, that may be detected when it is bound to a protein, is competed for that binding by the presence of a substance that itself binds to that protein.

iv) Screening Assays for Antagonists or Agonists of the Physical Interaction of BASP1, GID2, GID3 or GID4 Protein with a Second Protein

BASP1, GID2, GID3 or GID4 protein isolated and produced, described above, is contacted with substances present in the libraries, then a second protein that interacts with BASP1, GID2, GID3 or GID4 protein (described above) is added, in an assay such that the BASP1, GID2, GID3 or GID4 bound to second protein may be discriminated from the free proteins, and thereby a substance that increases the physical interaction of the two proteins (potential agonist) or decreases it (potential antagonist) is selected. For example, alterations in biophysical properties of the proteins associated with protein-protein binding, as described above, may be used to detect physical interaction; or a competition type of assay with antibody or a mimetic; or an altered biochemical activity of a second protein bound to BASP1, GID2, GID3 or GID4 that is suitable for a high throughput assay.

### Example 14

Assessment of the Activity of Isolated Antagonists or Agonists of BASP1, GID2, GID3 or GID4 Expression or Activity Identified in Screening Assays

The activity of antagonists and agonists is tested in any of a number of different assays to reveal properties of specificity or toxicity or utility:
i) Isolated dendritic cells or cells of similar function or cell lines as described above or unrelated cells or cell lines are contacted with increasing concentrations of the antagonist or agonist that is being tested, and the level of BASP1, GID2, GID3 or GID4 mRNA or protein expressed is determined by any of the methods described above and compared with that of untreated cells, and cells treated with antisense oligonucleotides and mismatch control antisense oligonucleotides as described above. Effects of the antagonist or agonist upon the expression of BASP1, GID2, GID3 or GID4 mRNA or protein are compared to that upon the expression of a second unrelated cellular gene.
ii) Isolated dendritic cells or cells of similar function or cell lines described above are contacted with increasing concentrations of the antagonist or agonist that is being tested, and the level of expressed activation markers dependent upon BASP1, GID2, GID3 or GID4 expression as described above are determined by any of the methods described above and compared with that of untreated cells, and cells treated with antisense oligonucleotides and mismatch antisense oligonucleotides as described above.
iii) Isolated dendritic cells or cells of similar function or cell lines described above are contacted with increasing concentrations of the antagonist or agonist that is being tested, and the level of expressed activation markers dependent upon specific physical interaction of BASP1, GID2, GID3 or GID4 with second cellular proteins as described above are determined by any of the methods described above and compared with that of untreated cells, and cells treated with interaction-interference polypeptides and mismatch control polypeptides as described above.
iv) Isolated dendritic cells or cells of similar function are contacted with increasing concentrations of the antagonist or agonist that is being tested, and the effect on cellular migration is determined as described above, and compared with that of untreated cells, cells treated with antisense oligonucleotides and mismatch antisense oligonucleotides as described above, and cells treated with interaction-interference polypeptides and mismatch control polypeptides as described above.
v) Isolated dendritic cells or cells of similar function or cell lines as described above are contacted with increasing concentrations of the antagonist or agonist that is being tested, and the effect on T cell activation capacity of the cells is determined as described above, and compared with that of untreated cells, cells treated with antisense oligonucleotides and mismatch antisense oligonucleotides as described above, and cells treated with interaction-interference polypeptides and mismatch control polypeptides as described above.
vi) Isolated dendritc cells or cells of similar function or cell lines as described above or unrelated cells or cell lines are contacted with increasing concentrations of the antagonist or agonist that is being tested, and the effect on cellular replication or viability is determined, as e.g. by the incorporation of radioactive or fluorogenic precursors of DNA synthesis, or e.g. the exclusion of colored or fluorescent dyes indicative of viability or membrane integrity, or e.g. by using commercially available kits based upon specific antibodies reacting with cellular components of dying cells.
vii) Isolated dendritic cells or cells of similar function or cell lines as described above or unrelated cells or cell lines are contacted with increasing concentrations of the antagonist or agonist that is being tested, and the effects on gene expression profiles is determined by DNA microarray hybridization techniques, and compared with those of cells treated with BASP1, GID2, GID3 or GID4 antisense oligonucleotides and mismatch control oligonucleotides, or interaction-interference polypeptides and mismatch control interaction-interference polypeptides, or antagonists or agonists of the activity of a second cellular gene unrelated to BASP1, GID2, GID3 or GID4.
viii) Isolated dendritic cells or cells of similar function or cell lines as described above or unrelated cells or cell lines are contacted with increasing concentrations of the antagonist or agonist that is being tested, and the effects on the profile of cellular proteins expressed and the state of those proteins is determined by proteomics techniques, and compared with those of cells treated with BASP1, GID2, GID3 or GID4 antisense oligonucleotides and mismatch control oligonucleotides, or interaction-interference polypeptides and mismatch control interaction-interference polypeptides, or antagonists or agonists of the activity of a second cellular gene unrelated to BASP1, GID2, GID3 or GID4.
ix) Isolated dendritic cells or cells of similar function or cell lines as described above that contain a recombinant reporter gene expression vector as described above are contacted with increasing concentrations of the antagonist or agonist that is being tested, and the effects on the activity of the BASP1, GID2, GID3 or GID4 promoter sequences contained in the reporter vector are measured as described above and compared with those obtained with a second reporter vector containing the promoter of a second cellular gene unrelated to BASP1, GID2, GID3 or GID4, and with untreated cells containing either of the said reporter vectors, and with cells containing either of the said reporter vectors that are treated with antagonists or agonists of the expression of a second cellular gene that is unrelated to BASP1, GID2, GID3 or GID4.
x) Purified BASP1, GID2, GID3 or GID4 protein and a second cellular interacting protein as described above are contacted with increasing concentrations of the antagonist or agonist that is being tested, and the effects on protein-protein interaction are determined by any of the methods described above and compared with those seen in the absense of treatment, with those seen in the presence of purified interaction-interference polypeptides and mismatch control interaction-interference polypeptides, or with those seen with treated and untreated mutated interaction-defective BASP1, GID2, GID3 or GID4 protein described above and a second interacting protein, or with any of the said purified proteins treated with antagonists or agonists of the interaction of proteins that are unrelated to the BASP1, GID2, GID3 or GID4 protein.
xi) Isolated dendritc cells or cells of similar function, or tissues that contain said cells, that are either untreated or treated with increasing concentrations of the antagonist or agonist that is being tested, are grafted into a suitable laboratory animal permissive for graft transplantation (e.g. SCID mice, e.g. irradiated animals) used in pharmaceutical testing that is subsequently challenged by an immune stimulus and the effects of the test substances on subsequent immunological reactions are determined, and compared with those seen with isolated cells or tissues treated with antisense oligonucleotides and mismatch antisense oligonucleotides as described above, or cells or tissues treated with interaction-interference polypeptides and mismatch control polypeptides as described above, or cells or tissues treated with unrelated antagonists or agonists as described above.
xii) Isolated dendritic cells or cells of similar function, or tissues that contain said cells are grafted into a suitable laboratory animal permissive for graft transplantation (e.g. SCID mice, e.g. irradiated animals) used in pharmaceutical testing, that is subsequently challenged by an immune stimulus and either untreated or treated with increasing concentrations of the antagonist or agonist that is being tested, and the effects of the test substances on subsequent immunological reactions are determined, and compared with those seen in the absense of pharmaceutical treatment.
xiii) Isolated dendritc cells or cells of similar function, or tissues that contain said cells, that are obtained from a suitable laboratory animal used in pharmaceutical testing, and that express the endogenous orthologue of that species of the BASP1, GID2, GID3 or GID4 gene, that are either untreated or treated with increasing concentrations of the antagonist or agonist that is being tested, are grafted into said animal that is subsequently challenged by an immune stimulus and the effects on subsequence immunological reactions are determined, and compared with those seen with isolated cells or tissues treated with antisense oligonucleotides and mismatch antisense oligonucleotides isolated for that orthologue as described above, or cells or tissues treated with interaction-interference polypeptides and mismatch control polypeptides isolated for that orthologue as described above, or cells or tissues treated with unrelated antagonists or agonists as described above.
xiv) Isolated dendritic cells or cells or suitable cell lines of similar function, that are obtained from a suitable laboratory animal used in pharmaceutical testing, and that have been engineered or treated such that they express a form of the orthologue of that species of the BASP1, GID2, GID3 or GID4 gene that corresponds in functional properties or properties of utility to any of the variants and or recombinants of the BASP1, GID2, GID3 or GID4 gene of humans, that are produced and introduced by any of the pertinent methods described and according to the invention, that are either untreated or treated with increasing concentrations of the antagonist or agonist that is being tested, are grafted into said animal that is subsequently challenged by an immune stimulus and the effects on subsequence immunological reactions are determined, and compared with those seen with isolated cells treated with antisense oligonucleotides and mismatch antisense oligonucleotides isolated for that orthologue as described above, or cells treated with interaction-interference polypeptides and mismatch control polypeptides isolated for that orthologue as described above, or cells or tissues treated with unrelated antagonists or agonists as described above.
xv) Isolated dendritic cells or cells or cell lines of similar function that are obtained from a suitable laboratory animal used in pharmaceutical testing, and that have been engineered or treated such that they express the human BASP1, GID2, GID3 or GID4 gene or a variant or recombinant form of it that comprises altered functional properties or properties of utility according to the invention, that are produced and introduced by any of the pertinent methods described and according to the invention, that are either untreated or treated with increasing concentrations of the antagonist or agonist that is being tested, are grafted into said animal that is subsequently challenged by an immune stimulus and the effects on subsequence immunological reactions are determined, and compared with those seen with isolated cells or tissues treated with antisense oligonudeotides and mismatch antisense oligonucleotides as described above.
xvi) Isolated dendritic cells or cells or cell lines of similar function that are obtained from a suitable laboratory animal used in pharmaceutical testing, and that have been engineered or treated such that they express a recombinant reporter gene vector whose expression is dependent upon the BASP1, GID2, GID3 or GID4 promoter sequences as described, or upon the promoter of the orthologous BASP1, GID2, GID3 or GID4 gene of the laboratory animal used in testing that is produced by the methods described and according to the invention, that are either untreated or treated with increasing concentrations of the antagonist or agonist that is being tested, are grafted into said animal that is subsequently challenged by an immune stimulus and the effects on the activity of the promoter contained within the recombinant reporter vector are determined, and compared with those seen with untreated cells or cells treated with antagonists or agonists of the expression of a second gene unrelated to BASP1, GID2, GID3 or GID4.
xvii) Laboratory animals commonly used in pharmaceutical testing, into which have been grafted any of the isolated dendritic cells or cells or cell lines of similar function as described, that have been untreated or treated to express any of the gene forms or complements or variants or recombinants of BASP1, GID2, GID3 or GID4 or othologues of BASP1, GID2, GID3 or GID4 or recombinant reporter gene vectors containing BASP1, GID2, GID3 or GID4 or appropriate orthologous promoter sequences as described, that are subsequently challenged by an immune stimulus, are treated with increasing concentrations of an antagonist or agonist that is being tested, and any of the effects described above are determined by any of the methods described above, and compared with those effects seen in animals treated or untreated with antisense oligonucleotides, interaction-interference polypeptides or antagonists or agonists of the expression or activity of a second gene that is unrelated to BASP1, GID2, GID3 or GID4.
xviii) Transgenic laboratory animals according to the invention that have been engineered to stably or conditionally contain and express the human BASP1, GID2, GID3 or GID4 gene or complement or variants or recombinants, or reporter gene vectors containing the human or orthologous BASP1, GID2, GID3 or GID4 promoter sequences, according to the invention, where the transgene is produced as an heterozygous or homozygous substitution of the endogenous BASP1, GID2, GID3 or GID4 orthologous gene or a substitution or insertion at a second locus, that are subsequently challenged by an immune stimulus, are treated with increasing concentrations of an antagonist or agonist that is being tested, and any of the effects described above are determined by any of the methods described above, and compared with those effects seen in animals treated or untreated with antisense oligonucleotides, interaction-interference polypeptides or antagonists or agonists of the expression or activity of a second gene that is unrelated to BASP1, GID2, GID3 or GID4.
xix) Genetically engineered laboratory animals which comprise homozygous deletions of the orthologous BASP1, GID2, GID3 or GID4 gene sequences or sufficient deletion to destroy BASP1, GID2, GID3 or GID4 activity according to the invention, that are challenged by an immune stimulus, are treated with increasing concentrations of the antagonist or agonist that is being tested, or isolated dendritc cells or cells of similar function from the animals are treated then re-introduced into the animals, and the effects on immunological reactions are determined and compared to those obtained in untreated animals or untreated cells re-introduced into the animals.
xx) Any of the genetic variants described above are produced in laboratory animals that have been inbred or engineered at other loci to effect specific immunological responses, e.g. within a genetic background that is highly susceptible to a specific challenge, e.g. to effect a more reproducible or penetrating immunological response following specific challenge, e.g. to effect immunological reactions in response to challenge that may be more characteristic of those observed in humans, and these variant animals are challenged by an immune stimulus and treated with increasing concentrations of the antagonist or agonist that is being tested, or isolated dendritic cells or cells of similar function from these animals or any of the isolated cells altered as described above are either treated or untreated by an immune stimulus or with increasing concentrations of antagonist or agonist and re-introduced or introduced into the animals, and the effects on immunological reactions are determined and compared to those obtained in untreated animals or untreated isolated cells that are introduced into the animals.

### Example 15

Overexpression of BASP1 in the Myeloid Cell Line U937

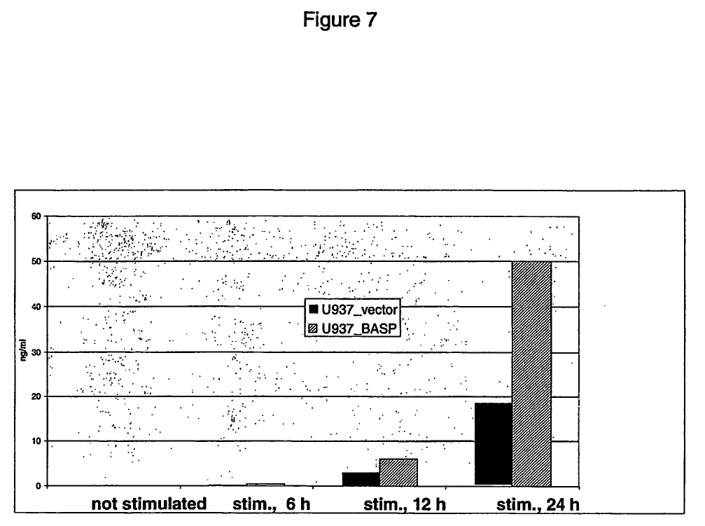

U 937 promyelocytic cells expressing C-terminally FLAG tagged BASP1 or empty vector are established by retroviral gene transduction and subsequent FACS cell sorting. Transfectants are stimulated with 10 μg/ml LPS at 1×10⁶ cells/ml. IL-8 secretion is measured by specific ELISA at the indicated time points (see e.g. FIG. 7). No change in the levels of TNF-α secretion were observed (data not shown).

These data indicate that the ectopic expression of BASP1 can modulate secretion of IL-8, an inflammatory cytokine. Therefore the interference with the BASP1 pathway could be considerated in disease such as inflammation and auto immune disease.

### Example 16

Overexpression of GID2.2 in Antigen-Presenting Cells

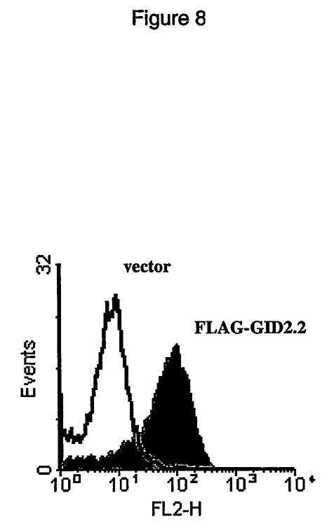

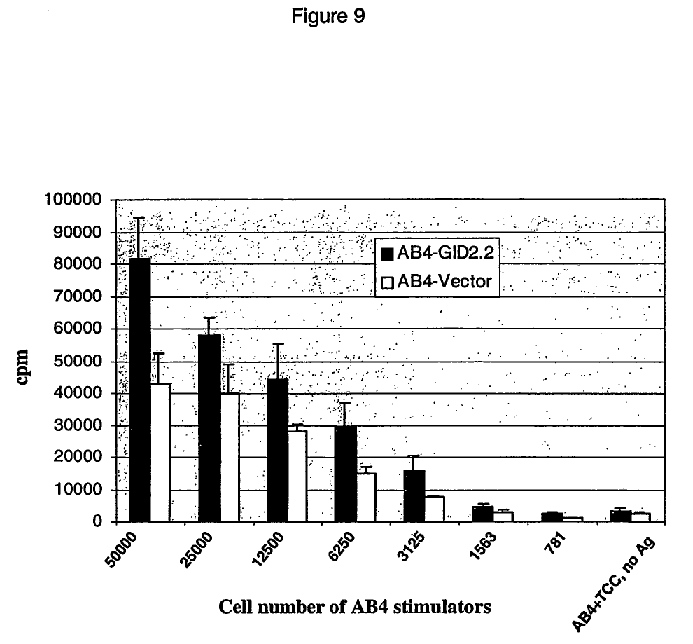

AB4 cells=EBV transformed B cells with a phenotype (e.g. surface markers like CD36, CD 40, CD83) and functionality (e.g. high expression of HLA-II antigen, ability to internalize, process and present the antigen as a complex with HLA-II) corresponding to dendritic cells expressing high levels of N-FLAG-GID2.2 or empty vector are established by retroviral transduction and subsequent FACS cell sorting (see e.g. FIG. 8). Vector or GID 2.2 transduced AB4 cells are loaded with DerP1 protein (=dermatophagoides protein of Pteronyssinus; major allergenic protein of the house-dust-mite) and used to stimulate the proliferation of a DerP1 specific T cell clone. Antigen-specific proliferation of the T cell clone is measured (see e.g. FIG. 9).

These data indicate that the ectopic expression of GID2.2 in antigen-presenting cells positively regulates T cell proliferation. Therefore interference with GID2.2 could be useful in disease where T cells are chronically stimulated such as in autoimmune disease, allergy and asthma.

1. An isolated gene expressed in dendritic cells of the immune system which is selected from BASP1, GID2, GID3 and GID4.

2. An isolated gene according to claim 1 comprising the polynucleotide, sequence of SEQ ID NO:1, or SEQ ID NO:2, or SEQ ID NO:3, or SEQ ID NO:5, or SEQ ID NO:7, or SEQ ID NO:8, or SEQ ID NO:10, or SEQ ID NO:11.

3. An isolated gene according to claim 1 encoding a polypeptide of sequence SEQ ID NO:4, or SEQ ID NO:6, or SEQ ID NO:9, or SEQ ID NO:12.

4. An isolated polypeptide encoded by a GID 2.1, GID 3 and/or GID 4 gene according to claim 1.

5. A vector comprising a gene according to claim 1 and/or a polypeptide according to claim 4 and/or SEQ ID NO:6.

6. An expression system comprising a gene according to claim 1, or a corresponding isolated promoter sequence as pan of a recombinant vector, wherein said expression system or pan thereof is capable of producing polypeptide encoded by a GID 2.1, GID 3 and/or GID 4 gene and/or a polypeptide according to SEQ ID NO:6 when said expression system or pan thereof is present in a compatible host cell.

7. An isolated host cell comprising an expression system according to claim 6.

8. A diagnostic kit for a disease or suspectability to a disease as described above, comprising:
(a) a gene encoding a sequence of e.g. of SEQ ID NO: 4, or SEQ ID NO: 6, or SEQ ID NO: 9, or SEQ ID NO: 12,
(b) a nucleotide sequence complementary to that of (a);
(c) a polypeptide; or
d) an antibody to a polypeptide.

9. An isolated antibody against a polypeptide as claimed in claim 4 or of SEQ ID NO:6.

10. An assay for screening to identify agonists or antagonists which decrease or enhance the production and or the biological activity of a polypeptide as claimed in claim 4, comprising
(a) a polypeptide as claimed in claim 4 or of SEQ ID NO:6;
(b) a host cell supporting an expression system comprising a polypeptide as claimed in claim 4 or of SEQ ID NO:6;
(c) a host cell supporting an expression system comprising the corresponding gene promoter and a recombinant polynucleotide transgene fused to a marker polynucleotide that expresses the resulting fusion polypeptide comprising a polynucleotide as claimed in claim 4 or of SEQ ID NO:6; or
(d) an antibody to a polypeptide as claimed in claim 4 or of SEQ ID NO:6.

11. An antagonist or an agonist of the expression and or the biological activity of a gene according to claim 1 that is characterized in that said antagonist or agonist can be provided by the following method steps:
A) contacting
(a) a polypeptide encoded by a GID 2.1, GID 3 and/or GID 4 gene or of SEQ ID NO:6;
(b) a host cell supporting an expression system comprising a polypeptide encoded by a GID 2.1, GID 3 and/or GID 4 gene or of SEQ ID NO:6;
(c) a host cell supporting an expression system comprising the corresponding gene promoter and a recombinant polynucleotide transgene fused to a marker polynucleotide that expresses the resulting fusion polypeptide comprising a polypeptide encoded by a GID 2.1, GID 3 and/or GID 4 gene or of SEQ ID NO:6; or
(d) an antibody to a polypeptide encoded by a GID 2.1, GID 3 and/or GID 4 gene or of SEQ ID NO:6 with a candidate compound,
B) determining the effect of the candidate compound on any of a), b), c) or d);
C) choosing an agonist or antagonist determined in step B).

12. An antagonist or an agonist according to claim 11 for use as a pharmaceutical.

13. A pharmaceutical composition comprising a pharmaceutically effective amount of an agonist or an antagonist according to claim 11 in combination with pharmaceutically acceptable carrier(s)/excipient(s).

14. A method of treating abnormal conditions related to both an excess of and insufficient level of expression of a gene according to claim 1; or related to both an excess of and insufficient activity of a polypeptide encoded by a GID 2.1, GID 3 and/or GID 4 gene or of SEQ ID NO:6, comprising administering a therapeutically effective amount of an agonist or antagonist wherein said antagonist or agonist can be provided by the following method steps:
A) contacting
(a) a polypeptide encoded by a GID 2.1, GID 3 and/or GID 4 gene or of SEQ ID NO:6:
(b) a host cell supporting an expression system comprising a polypeptide encoded by a GID 2.1, GID 3 and/or GID 4 gene or of SEQ ID NO:6;
(c) a host cell supporting an expression system comprising the corresponding gene promoter and a recombinant polynucleotide transgene fused to a marker polynucleotide that expresses the resulting fusion polypeptide comprising a polypeptide encoded by a GID 2.1. GID 3 and/or GID 4 gene or of SEQ ID NO:6; or
(d) an antibody to a polypeptide encoded by a GID 2.1. GID 3 and/or GID 4 gene or of SEQ ID NO:6 with a candidate compound.
B) determining the effect of the candidate compound on any of a), b), c) or d);
C) choosing an agonist or antagonist determined in step B);
to a subject in need of said treating.

15. A method according to claim 14 wherein abnormal conditions which may be treated include acute and chronic inflammatory diseases, including for example inflammatory skin diseases such as allergic and atopic dermatitis, and chronic inflammatory diseases whose symptoms appear in other tissues such as intestine and colon, lung and vascular tissue; e.g. such as abnormal immune reactivity in autoimmune diseases, or undesired immunological reactions associated with therapeutic treatment of diseases as in transplant rejection crises; e.g. immune hypo-reactivity or suppression in diseases such as cancer, and that associated with persistent viral or microbial infection.

16. A gene according to claim 1 expressed in DCs of the immune system and encoding a protein of the corresponding polypeptide sequence, whose expression in DCs is regulated by inflammatory stimuli, as a specific molecular target in DCs for therapeutic intervention in diseases in which immunological reactions are a primary component of etiology, progression and/or exacerbating symptoms associated with the disease.

17. A bispecific reagent which is other than an antibody, having the ability to bind to a polypeptide as claimed in claim 4 or of SEQ ID NO:6 and to a viral, bacterial or eukaryotic parasite or to a cancer cell.

18. A method for inducing an immunological response in a mammal that comprises inoculating said mammal with a polypeptide as claimed in claim 4 or of SEQ ID NO:6 in an amount sufficient to produce antibody and/or T cell immune response(s) to protect said mammal from diseases.

19. An immunological/vaccine formulation (composition) that, when introduced into a mammal, induces an immunological response in that mammal to a polypeptide as claimed in claim 4 or of SEQ ID NO 6, wherein said formulation comprises a polypeptide as claimed in claim 4 or of SEQ ID NO:6, or an expression vector comprising a polypeptide as claimed in claim 4 or of SEQ ID NO:6, or host cells comprising cells from said mammal that is treated that contain an expression vector comprising a polypeptide as claimed in claim 4 or of SEQ ID NO:6.
